(19) United States
(12) Patent Application Publication
(10) Pub. No.: US 2021/0369681 A1
(43) Pub. Date: Dec. 2, 2021

(54) Identification of PPM1D mutations as a novel biomarker for NAMPTi sensitivity

(71) Applicants: YALE UNIVERSITY, New Haven, CT (US); University of Iowa Research Foundation, Iowa City, IA (US)

(72) Inventors: Ranjit Bindra, New Haven, CT (US); Nathan FONS, Washington, DC (US); Charles M. BRENNER, Iowa City, IA (US)

(21) Appl. No.: 17/285,849

(22) PCT Filed: Oct. 22, 2019
(86) PCT No.: PCT/US2019/057386

§ 371 Date: Apr. 15, 2021

Related U.S. Application Data
(60) Provisional application No. 62/748,911, filed on Oct. 22, 2018.

(51) Int. Cl. A61K 31/422; G01N 33/68; A61K 45/06; A61K 31/4192; A61K 31/4406; A61K 31/444; A61K 31/437; A61K 31/455

(52) U.S. Cl. CPC A61K 31/422; G01N 33/6893; A61K 45/06; A61K 31/4192; G01N 2800/7028; A61K 31/444; A61K 31/437; A61K 31/455; G01N 2333/916; A61K 31/4406

(57) ABSTRACT

The present invention provides a method of treating cancer in a subject, the method comprising administering to the subject at least one nicotinamide phosphoribosyltransferase (NAMPT) inhibitor, thereby treating the cancer, wherein protein phosphatase Mg²⁺/Mn²⁺ dependent 1D (PPM1D) is elevated in the cancer.

### CROSS-REFERENCE TO RELATED APPLICATIONS

The present application claims priority under 35 U.S.C. § 119(e) to U.S. Provisional Patent Application No. 62/748,911 filed Oct. 22, 2018, which application is hereby incorporated by reference in its entirety herein.

### BACKGROUND OF THE INVENTION

The Protein Phosphatase Mg²⁺/Mn²⁺ Dependent 1D (PPM1D) gene, also known as Wip1, encodes a serine/threonine phosphatase which dephosphorylates numerous proteins primarily involved in the DNA damage response (DDR) and cellular checkpoint pathways. Since its discovery over 20 years ago, PPM1D has become a well-established oncogene, found amplified or over-expressed in a diverse range of cancers, including breast, ovarian, gastrointestinal, and brain cancers. Truncation mutations in the C-terminus of PPM1D were subsequently identified in a subset of cancers, most notably in pediatric gliomas, including diffuse intrinsic pontine glioma (DIPG). These mutations markedly enhance the protein stability of PPM1D, which similarly increases its phosphatase activity. Despite characterization of the cellular function of PPM1D, there remains much to be understood about its role in tumorigenesis. To compound this, there are no isogenic glial cell lines that contain PPM1D truncating mutations, limiting the ability to study their oncogenic role. Finally, while a number of PPM1D inhibitors have been developed as experimental tools, their in vitro success has yet to translate into the clinic. There is a need in the art for novel compounds and compositions that can be used to treat cancer. The present disclosure addresses this need.

### SUMMARY OF THE INVENTION

In one aspect, the invention provides a method of treating cancer in a subject, the method comprising administering to the subject at least one nicotinamide phosphoribosyltransferase (NAMPT) inhibitor, thereby treating the cancer, wherein protein phosphatase Mg²⁺/Mn²⁺ dependent 1D (PPM1D) is elevated in a biopsy sample obtained from the cancer in the subject.

In various embodiments, the method further comprises detecting an elevated level of PPM1D relative to a reference level, in a cancer cell sample obtained from the subject.

In various embodiments, the cancer comprises one or more mutations in the PPM1D gene.

In various embodiments, PPM1D comprises a C-terminal truncation mutation.

In various embodiments, the at least one NAMPT inhibitor is selected from the group consisting of OT-82, KPT-9274, FK866, GNE-618, LSN-3154567, FK866, STF31, GPP78, and STF118804.

In various embodiments, the cancer is breast, ovarian, gastrointestinal, brain cancer, medulloblastoma or pediatric glioma.

In various embodiments, the method further comprises administering to the subject at least one additional nicotinamide adenine dinucleotide (NAD) depleting treatment.

In various embodiments, the additional NAD depleting treatment is selected from the group consisting of temozolomide, etoposide, irinotecan and radiation therapy.

In various embodiments, the method further comprises administering supplemental nicotinamide to the subject.

In various embodiments, an effective amount of the NAMPT inhibitor is administered to the subject in a pharmaceutical composition comprising at least one pharmaceutically acceptable excipient.

In various embodiments, the subject is a mammal.

In various embodiments, the subject is a human.

### BRIEF DESCRIPTION OF THE DRAWINGS

The following detailed description of illustrative embodiments of the invention will be better understood when read in conjunction with the appended drawings. For the purpose of illustrating the invention, certain illustrative embodiments are shown in the drawings. It should be understood, however, that the invention is not limited to the precise arrangements and instrumentalities of the embodiments shown in the drawings.

FIGS. 1A-1J: PPM1D mutant immortalized human astrocytes are sensitive to NAMPT inhibitors. FIG. 1A: Previously identified (refs 8,9,10) PPM1D truncation mutations in pediatric HGGs (blue circles). CRISPR-modified mutations in human astrocytes shown in red arrows. FIG. 1B: Immunoblot of PPM1D full-length (full arrow) and truncated (arrowhead) protein expression across parental astrocytes (Par.), an isolated wild type astrocyte clone (WT iso.), and four different isolated CRISPR-modified, PPM1D-truncated (PPM1Dtrnc.) astrocytes. FIG. 1C: Immunoblot of PPM1D expression post cycloheximide (CHX) and MG132 treatment. FIG. 1D: Quantification of the experiment in c., (n=3 biologically independent experiments, * p<0.05, ** p<0.01 by Student's T test). FIG. 1E: Representative images of cellular 7H2AX foci, +/−treatment with 10 Gy ionizing radiation (IR). FIG. 1F: Quantification of 7H2AX foci in untreated, IR-treated, and concurrent IR plus 50 nM PPM1D inhibitor GSK2830371 treatment (PPM1Di); (n=4 biologically independent samples, ** p<0.001 by Student's T test). FIG. 1G: Calculated IC50 ratios (Parental/PPM1Dtrnc.) for a library of tested small molecule inhibitors. FIG. 1H: Viability assessment of wild type (Par. Astros. and WT iso.) and three PPM1Dtrnc. cell lines, 72 hrs post FK866 treatment (n=3 biologically independent samples). FIG. 1I: Calculated IC₅₀ values of parental (black highlight) and PPM1Dtrnc. (red highlight) astrocytes for different NAMPT inhibitors; length of bar represents selectivity window of the given drug for PPM1D mutant cells (n=2 biologically independent experiments). FIG. 1J: Viability analysis of cell lines in response to 72 hrs of FK866 treatment (n=3 biologically independent samples). All error bars represent standard deviation of the mean.

FIGS. 2A-2K Mutant PPM1D-induced NAPRT deficiency drives sensitivity to NAMPT inhibition. FIG. 2A: Graphic model of enzymes and metabolites involved in NAD biosynthesis. NA: nicotinic acid; NAAD: nicotinic acid adenine dinucleotide; NAD: nicotinamide adenine dinucleotide; NADP: nicotinamide adenine dinucleotide phosphate; NAM: nicotinamide; NAMN: nicotinic acid mononucleotide; NAR: nicotinic acid riboside; NMN: nicotinamide mononucleotide; NR: nicotinamide riboside; QA: quinolinic acid; Trp: tryptophan. FIG. 2B: Heatmap of NAD-related metabolites in parental and two different PPM1Dtrnc. astrocyte cell lines. FIG. 2C: NAD quantification in wild type and PPM1Dtrnc. astrocytes (n=3 biological independent samples, **** p<0.0001 by Student's T test). FIG. 2D: Relative fold change in NAD levels post 10 nM FK866 treatment (n=3 biological independent samples, *** p<0.001 by Student's T test). FIG. 2E: Bliss 3D surface plot modelling the antagonistic effects of NR on FK866 treatment in PPM1Dtrnc. astrocytes. FIG. 2F: Cell viability analysis of parental astrocytes treated with either scrambled control (scrbl) or NAPRT siRNAs, followed by treatment with FK866 (n=2 biological independent samples, **** p<0.0001 by Student's T test). FIG. 2G: Immunoblot of isogenic astrocytes., and astrocytes stably-overexpressing WT and mutant PPM1D (OEFL and OEtrnc., respectively). Full length (full arrow), CRISPR-modified (black arrowhead), and ectopic mutant (white arrowhead) sizes of PPM1D displayed. FIG. 2H: Viability assessment of isogenic astrocytes and stable NAPRT-expressing PPM1Dtrnc. astrocytes (PPM1Dtrnc.+NAPRT), to FK866 treatment (n=4 biological independent samples, *** p<0.001, **** p<0.0001 by Student's T test). FIG. 2I: Immunoblot of previously described wild type and PPM1D mutant astrocytes, and patient-derived, SU-DIPG cell lines. FIG. 2J: Viability assessment of SU-DIPG cell lines post 120 hr treatment with FK866 (n=3 biological independent samples). FIG. 2K: Representative images from spheroid cultures in j., untreated or treated with 10 nM FK866. All error bars represent standard deviation of the mean.

FIGS. 3A-3F Epigenetic events silence NAPRT expression in PPM1D mutant glioma models. FIG. 3A: Quantification of NAPRT transcript levels via qPCR, in wild type (grey) and mutant PPM1D-expressing (red) astrocytes and DIPG cell lines (n=3 biological independent samples, ** p<0.01, *** p<0.001 by Student's T test). FIG. 3B: Chromatin Immunoprecipitation (ChTP) of common histone 3 modifications at the NAPRT promoter; quantified as fold enrichment over IgG control (n=4 biological independent samples, ** p<0.01, **** p<0.0001 by Student's T test). FIG. 3C: Quantification of methylated DNA (5-meC), and hydroxymethylated DNA (5 hmC), immunoprecipitated from the NAPRT promoter (n=2 biological independent samples, ** p<0.01 by Student's T test). FIG. 3D: Sequencing chromatograms of the NAPRT promoter within astrocytes and SU-DIPG cell lines after bisulfite conversion; arrows indicate potential CpG methylation sites. FIG. 3E: Heatmap and clustering analysis of the 390 most significant variable Infinium Methylation EPIC array probes, across different astrocyte and DIPG models. FIG. 3F: Heatmap and hierarchical clustering analysis of methylation array probes located within NAPRT CpG island promoter region. All error bars represent 95% confidence intervals about the mean.

FIGS. 4A-4D: NAMPT inhibitors are effective in vivo agents against PPM1D mutant xenografts. FIG. 4A: Fold change in tumor growth for serially-transplanted PPM1Dtrnc. xenografts in NSG mice treated with vehicle or 20 mg/kg FK866 BID for 3 cycles of: four days on, followed by three days off (n=7 animals, *** p<0.001 by Mann-Whitney U test, error bars represent standard deviation of the mean). Arrows indicate initiation of treatment cycle. FIG. 4B: Kaplan-Meier plot of xenograft tumor growth from a., with arrows indicating initiation of treatment cycle (p<0.0001 by Log rank (Mantel-Cox) test). FIG. 4C: NAPRT expression levels for PNOC003 DIPG cohort (31) samples. FIG. 4D: Model depicting the mechanism of mutant PPM1D-induced dependence on NAMPT for NAD production, and synthetic lethality with NAMPT inhibitors, such as FK866.

FIGS. 5A-5G: PPM1D mutant astrocytes are sensitive to NAMPT inhibitors. FIG. 5A: Sequencing chromatograms within a region of PPM1D exon 6 from parental and PPM1Dtrnc. cell lines. FIG. 5B: Immunoblot of parental and PPM1Dtrnc. cell lines in response to radiation. Full length (full arrow) and CRISPR-modified (arrowhead) sizes of PPM1D displayed. FIG. 5C: Quantification of γH2AX foci post radiation (IR) (n=4 independent samples). FIG. 5D: Viability assessments of cell lines after 72 hr treatment with three different NAMPT inhibitors (GPP78, STF118804, and STF31) (n=3 independent samples). FIG. 5E: Quantification of PPM1D transcript levels in astrocyte cell lines (n=4 independent samples). FIG. 5F: Immunoblot of astrocytes with stable expression of wild type (OEFL) or mutant (OEtrnc.) PPM1D. Full length (full arrow), CRISPR-edited (black arrowhead), and ectopically-expressed mutant protein (white arrowhead) sizes of PPM1D are displayed. FIG. 5G: Representative wells of H33342-stained nuclei from parental and mutant astrocytes, 72 hrs post DMSO or FK866 treatment. Error bars represent standard deviation of the mean.

FIGS. 6A-6L: NAD metabolome depression in PPM1Dtrnc. astrocytes results in NAMPT inhibitor sensitivity. FIG. 6A: NADP quantification in parental and PPM1Dtrnc. astrocytes (n=3 independent samples, *** p<0.001 by Student's T test). FIG. 6B: Relative fold change in NADP levels after treatment with 10 nM FK866 for 24 hrs (n=3 independent samples, ** p<0.01 by Student's T test). FIG. 6C: NAD quantification after exogenous addition of 50 μM nicotinamide riboside (NR) for 24 hrs (n=3 independent samples, * p<0.05, ** p<0.01 by Student's T test). FIG. 6D: Normalized NAD levels in astrocytes after 24 hr treatment with 10 nM FK866 and indicated doses of NR (n=2 independent samples). FIG. 6E: Bliss model matrix for the antagonistic effects of NR on FK866 treatment in PPM1Dtrnc. astrocytes. FIG. 6F: Viability assessment of PPM1Dtrnc. astrocytes after 72 hr concurrent FK866 and NR treatment. FIG. 6G and FIG. 6J: Bliss 3D surface plots modelling the antagonistic effects of NAM (FIG. 6G) or NA (FIG. 6J) on FK866 treatment in PPM1Dtrnc. astrocytes. FIGS. 6H and 6K: Bliss model matrices for the antagonistic effects of NAM (FIG. 6H) or NA (FIG. 6K) on FK866 treatment in PPM1Dtrnc. FIG. 6I and FIG. 6L: Viability assessment of PPM1Dtrnc. astrocytes after 72 hr concurrent treatment of FK866 with NAM (FIG. 6I) or NA (FIG. 6L). Error bars represent standard deviation of the mean.

FIGS. 7A-7E: NAPRT deficiency drives sensitivity of PPM1D mutant astrocytes to NAMPT inhibitors. FIG. 7A: Normalized viability of parental (left) and PPM1Dtrnc. (right) astrocytes to FK866 treatment after transfection with a panel of siRNAs targeting NAD biosynthesis-related enzymes (n=2 independent samples). FIG. 7B: Immunoblot of NAPRT protein level after treatment with different NAPRT-targeted siRNAs. FIG. 7C: Viability analysis of cell lines in b., treated with FK866 for 72 hrs (n=4 independent samples). FIG. 7D: Immunoblot of parental and PPM1Dtrnc. astrocytes+/−stable expression of NAPRT. FIG. 7E: Viability assessment Par. Astros., PPM1Dtrncs., and a NAPRT-expressing PPM1Dtrnc. (PPM1Dtrnc.+NAPRT) cell line upon 72 hr FK866 treatment (n=4 independent samples). Error bars represent standard deviation of the mean.

FIGS. 8A-8C: Patient-derived SU-DIPG-XXXV spheroid cell line possesses a truncating PPM1D mutation and is sensitive to NAMPT inhibitors. FIG. 8A: Sequencing chromatograms within a region of PPM1D exon 6, from SU-DIPG-IV, XIII, and XVII spheroid cell lines. FIG. 8B: Chromatogram of PPM1D-truncating mutation in SU-DIPG-XXXV. FIG. 8C: Viability assessments of SU-DIPG spheroids to FK866 in nicotinic acid (NA) containing (+NA) or NA lacking (−NA) culture media (n=3 independent samples). Error bars represent standard deviation of the mean.

FIGS. 9A-9E: U2OS and MCF7 cell lines contain PPM1D alterations, silence NAPRT transcription, and are sensitive to NAMPT inhibitors. FIG. 9A: Immunoblot of isogenic astrocytes, U2OS, and MCF7 cell lines. FIG. 9B and FIG. 9C: Normalized mRNA expression of PPM1D (FIG. 9B) and NAPRT (FIG. 9C) in cell panel from a (n=4 independent samples). Error bars represent 95% Confidence Interval about the mean. (FIG. 9D) Sequencing chromatograms of the NAPRT promoter within U2OS and MCF7 cell lines after bisulfite conversion; arrows indicate potential CpG methylation sites. (FIG. 9E) Viability assessment of isogenic astrocytes, U2OS, and MCF7 cell lines after 96 hr treatment with FK866 (n=3 independent samples). Error bars represent standard deviation of the mean.

FIGS. 10A-10E: DIPG model cell lines with PPM1D mutations have reduced NAPRT expression and maintain p53 expression. FIG. 10A Table depicting mutational status of patient-derived DIPG cell lines in FIG. 3E; ND indicates no data available. FIG. 10B: NAPRT expression levels of model DIPG cell lines. FIG. 10C: Immunoblot of select astrocyte and DIPG cell lines for NAPRT and H3K27M expression. FIG. 10D: Viability of HSJD-DIPG-007 cell line after 120 hr of treatment with FK866 (n=5 independent samples). Error bars represent standard deviation of the mean. FIG. 10E: Immunoblot of DIPG cell line panel for p53 and H3K27M expression.

FIGS. 11A-11E: Mutant PPM1D-induced hypermethylation is distinct from G-CIMP found in IDH1 mutant astrocytes. FIG. 11A and FIG. 11B: Hierarchical clustering of the top 2% of significantly variable methylation probes in astrocyte (FIG. 11A) and DIPG (FIG. 111B) cell lines. (FIG. 11C) Comparison of top 2% significantly variable CpG island probesets in PPM1D mutant- and IDH1 mutant astrocytes. FIG. 11D: Normalized levels of global 5-hydroxymethylcytosine in WT and PPM1D mutant astrocytes (n=4 independent samples). Error bars represent 95% Confidence Interval about the mean. FIG. 11E: Immunoblot of parental and PPM1Dtrnc, astrocytes after treatment with varying doses of decitabine (DCT) or azacytidine (azaC) for 72 hrs.

FIGS. 12A-12E: In vivo efficacy of NAMPT inhibitors in PPM1D mutant tumors. FIG. 12A: PPM1Dtrnc. tumor burden as a measure of bioluminescence imaging (BLI) signal, in NOD scid gamma mice treated with vehicle or 20 mg/kg FK866 BID for 3 four day cycles as indicated by arrows (n=10 independent animals, error bars represent SE, ** p<0.01, *** p<0.001 by Mann-Whitney U test). FIG. 12B Representative BLI images of vehicle and FK866-treated mice over course of treatment. FIG. 12C: Tumor mass measurements, from extracted tumors in a., 2 months post injection (n=14 independent tumors, **** p<0.0001 by Student's T test). FIG. 12D: Comparison of BLI signal intensity between PPM1Dtrnc. cell line xenografts and serially-transplanted PPM1D mutant xenografts, 12 days post injection (n=17 independent tumors, ** p<0.01 by Student's T test). Error bars represent standard deviation of the mean. FIG. 12E: Representative BLI images of serially-transplanted PPM1D mutant xenografts before or after 3 weeks of indicated treatment.

FIGS. 13A-13E: Applicability of NAMPT inhibitors for the treatment of PPM1D mutant, non-glioma tumors. FIG. 13A: Tumor volume measurements of vehicle or FK866-treated athymic nude mice harboring U2OS cell line xenografts. FK866 treatment consisted of 20 mg/kg BID for 3 four day weekly cycles, indicated by arrows (n=15 independent animals, **** p<0.0001 by Mann-Whitney U test). Error bars represent standard deviation of the mean. FIG. 13B: Percent change in body mass, measured for each mouse during the duration of treatment described in FIG. 13A. FIG. 13C: NAPRT and PPM1D expression levels from PNOC003 DIPG cohort (31) tumor samples. FIG. 13D: Comparison of NAPRT expression levels in wild type and PPM1D mutant DIPG tumors from the cohort in FIG. 13C. FIG. 13E: Comparison of NAPRT expression levels in PPM1D high and low expressing tumors, in cancer subtypes commonly found to have amplification of PPM1D (left); with histograms of PPM1D expression (right). * p<0.05 ** p<0.01 by Student's T test.

### DETAILED DESCRIPTION OF THE INVENTION

The present invention relates in part to the unexpected discovery that cancers with elevated levels of PPM1D activity may be effectively treated with NAMPT inhibitors. Without wishing to be limited by theory, the data presented herein indicates that this may be due to the shutdown of one of the major pathways for the production of NAD in the cell by silencing nicotinic acid phosphoribosyltransferase (NAPRT). This makes the NAMPT pathway for production NAD critical to cell survival and therefore inhibition of this pathway may selectively kill cancer cells that cannot rely on NAPRT associated NAD production while sparing non-cancerous cells which can.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section.

Generally, the nomenclature used herein and the laboratory procedures in cell culture, molecular genetics, pharmacology and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

A disease or disorder is “alleviated” if the severity or frequency of at least one sign or symptom of the disease or disorder experienced by a patient is reduced.

As used herein, the terms “analog,” “analogue,” or “derivative” are meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

As used herein, the term “biopsy sample” means any type of sample obtained from a subject by biopsy or any sample containing tissue, cells or fluid associated with a cancerous growth in a subject.

The term “elevated” as used herein when applied to a gene, protein or chemical reaction means that the expression, activity or concentration of the gene, protein or reaction is higher compared to an appropriate control.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the terms “nicotinamide adenine dinucleotide depleting treatment” or “NAD depleting treatment” mean treatments that reduce the level of nicotinamide adenine dinucleotide (NAD) either globally in the subject or locally. In various embodiments, the NAD depleting therapy may be in combination with the administration of temozolomide and/or radiation therapy.

As used herein, the terms “nicotinamide phosphoribosyltransferase” or “NAMPT” refer to the nicotinamide phosphoribosyltransferase gene or protein having UniProt accession number P43490 and having the amino acid sequence:

```
SEQ ID NO: 15
                10 20 30 40
MNPAAEAEFN ILLATDSYKV THYKQYPPNT SKVYSYFECR
                50 60 70 80
EKKTENSKLR KVKYEETVFY GLQYILNKYL KGKVVTKEKI
                90 100 110 120
QEAKDVYKEH FQDDVFNEKG WNYILEKYDG HLPIEIKAVP
              130 140 150 160
EGFVIPRGNV LFTVENTDPE CYWLTNWIET ILVQSWYPIT
              170 180 190 200
VATNSREQKK ILAKYLLETS GNLDGLEYKL HDFGYRGVSS
              210 220 230 240
QETAGIGASA HLVNFKGTDT VAGLALIKKY YGTKDPVPGY
              250 260 270 280
SVPAAEHSTI TAWGKDHEKD AFEHIVTQFS SVPVSVVSDS
              290 300 310 320
YDIYNACEKI WGEDLRHLIV SRSTQAPLII RPDSGNPLDT
              330 340 350 360
VLKVLEILGK KFPVTENSKG YKLLPPYLRV IQGDGVDINT
              370 380 390 400
LQEIVEGMKQ KMWSIENIAF GSGGGLLQKL TRDLLNCSFK
              410 420 430 440
CSYVVTNGLG INVFKDPVAD PNKRSKKGRL SLHRTPAGNF
              450 460 470 480
VTLEEGKGDL EEYGQDLLHT VFKNGKVTKS YSFDEIRKNA
              490
QLNIELEAAH H
```

for the human homolog.

As used herein, the terms “nicotinamide phosphoribosyltransferase inhibitor” or “NAMPT inhibitor” refer to any agent that inhibits NAMPT. In various embodiments, the NAMPT inhibitor may be nucleic acid based inhibitor, such as a small interfering RNA or antisense oligonucleotide. In various embodiments, the NAMPT inhibitor may be a small molecule.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

As used herein, the terms “protein phosphatase Mg²⁺/Mn²⁺ dependent 1D” or “PPM1D” means the protein phosphatase Mg²⁺/Mn²⁺ dependent 1D gene or protein having UniProt Accession number A0A0S2Z4M2 and having amino acid sequences:

```
SEQ ID NO: 16:
                10 20 30 40
MAGLYSLGVS VFSDQGGRKY MEDVTQIVVE PEPTAEEKPS
                50 60 70 80
PRRSLSQPLP PRPSPAALPG GEVSGKGPAV AAREARDPLP
                90 100 110 120
DAGASPAPSR CCRRRSSVAF FAVCDGHGGR EAAQFAREHL
              130 140 150 160
WGFIKKQKGF TSSEPAKVCA AIRKGFLACH LAMWKKLAEW
              170 180 190 200
PKTMTGLPST SGTTASVVII RGMKMYVAHV GDSGVVLGIQ
              210 220 230 240
DDPKDDFVRA VEVTQDHKPE LPKERERIEG LGGSVMNKSG
              250 260 270 280
VNRVVWKRPR LTHNGPVRRS TVIDQIPFLA VARALGDLWS
              290 300 310 320
YDFFSGEFVV SPEPDTSVHT LDPQKHKYII LGSDGLWNMI
              330 340 350 360
PPQDAISMCQ DQEEKKYLMG EHGQSCAKML VNRALGRWRQ
              370 380 390 400
RMLRADNTSA IVICISPEVD NQGNFTNEDE LYLNLTDSPS
              410 420 430 440
YNSQETCVMT PSPCSTPPVK SLEEDPWPRV NSKDHIPALV
              450 460 470 480
RSNAFSENFL EVSAEIAREN VQGVVIPSKD PEPLEENCAK
              490 500 510 520
ALTLRIHDSL NNSLPIGLVP TNSTNTVMDQ KNLKMSTPGQ
              530 540 550 560
MKAQEIERTP PTNFKRTLEE SNSGPLMKKH RRNGLSRSSG
              570 580 590 600
AQPASLPTTS QRKNSVKLTM RRRLRGQKKI GNPLLHQHRK
TVCVC
```

for the human homolog.

By the term “specifically binds,” as used herein, is meant a molecule, such as an antibody, which recognizes and binds to another molecule or feature, but does not substantially recognize or bind other molecules or features in a sample.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, the term “wild-type” refers to the genotype and phenotype that is characteristic of most of the members of a species occurring naturally and contrasting with the genotype and phenotype of a mutant.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of Treatment

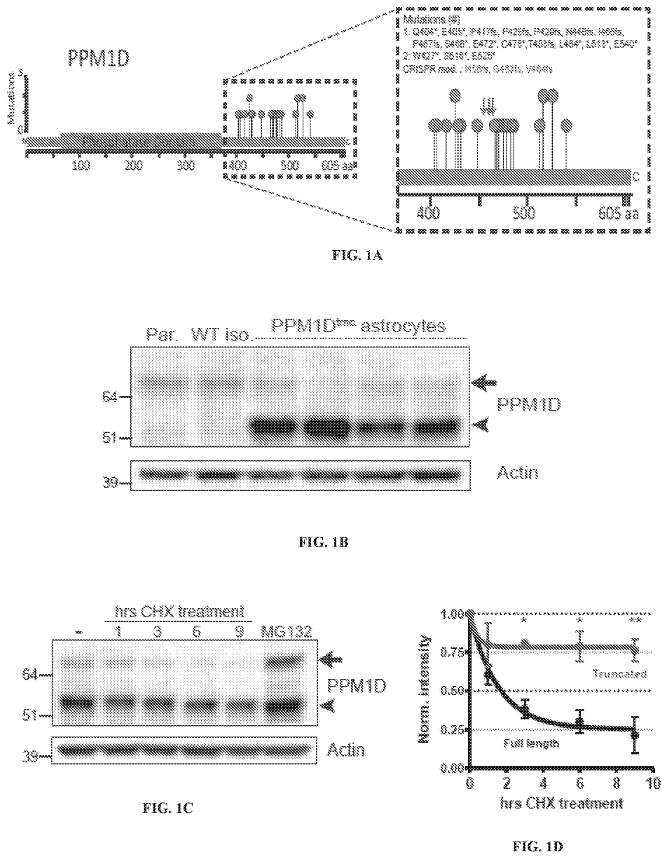

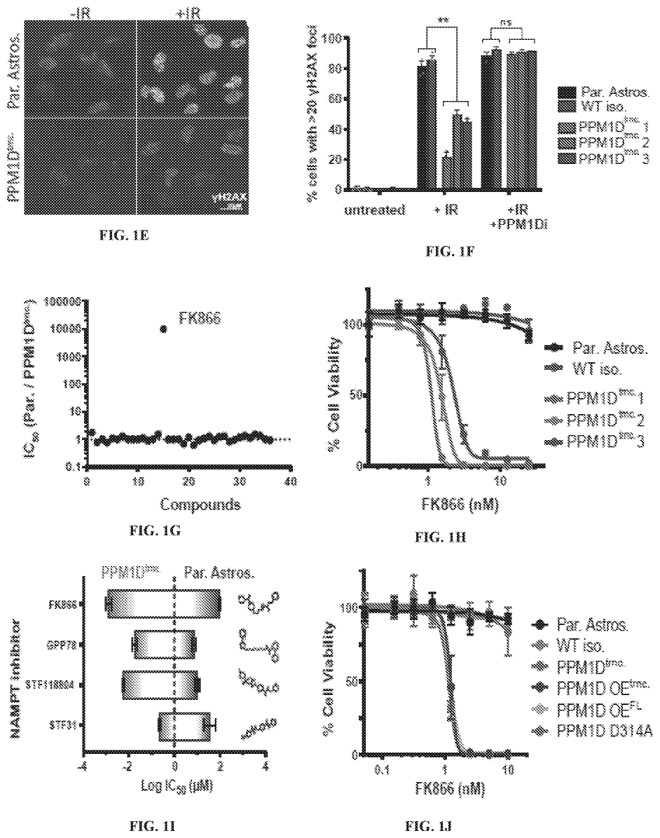

Without wishing to be limited by theory, the invention is based in part on the unexpected discovery that, as shown in Example 1 and FIGS. 1A-4D, cancers exhibiting an elevated level protein phosphatase Mg²⁺/Mn²⁺ dependent 1D (PPM1D) are sensitized to treatment with nicotinamide phosphoribosyltransferase (NAMPT) inhibitors. Accordingly, in one aspect the invention provides a method of treating cancer in a subject, the method comprising administering to the subject an effective amount of at least one NAMPT inhibitor, thereby treating the cancer, wherein PPM1D is elevated is elevated in a biopsy sample obtained from the cancer in the subject.

The precise reason that PPM1D activity is elevated is not critical to the practice of various embodiments of the invention. In various embodiments, PPM1D activity may be heightened relative to controls because the concentration of PPM1D protein is higher. In some embodiments this is due to increased production of PPM1D and in other embodiments this is due to decreased degradation of PPM1D.

Certain mutations in PPM1D generate a hyper-stable form of the protein with the net result that PPM1D activity is heightened within the cancer cell. The nature of the mutation that generates hyper-stable PPM1D is not critical. This variant has been associated with a C-terminal truncation mutation in PPM1D. Accordingly, in various embodiments, PPM1D comprise a C-terminal truncation mutation.

In various embodiments, the method further comprises detecting an elevated level of PPM1D in a biopsy sample obtained from the subject. The sample may be obtained using any means known in the art, by way of non-limiting example, by biopsy. As a skilled person will realize, there are a variety of ways to determine that PPM1D is elevated in the cancer of the subject or a subset of the cancer cells or the tumor or other cancerous growth. All of these are contemplated and included in the methods of the invention. By way of non-limiting example, the PPM1D gene may be amplified, the level of PPM1D mRNA may be amplified or PPM1D protein stability may be enhanced.

Various NAMPT inhibitors may be utilized in various embodiments of the invention. In various embodiments, one or more NAMPT inhibitor s are selected from the group consisting of OT-82, KPT-9274, GNE-618, LSN-3154567, FK866, STF31, GPP78, STF118804, GMX-1778, GNE-617 and A-1293201. Other suitable NAMPT inhibitors are disclosed in U.S. Publication No. 2017/0174704 which is hereby incorporated by reference. Structures for these compounds are shown below.

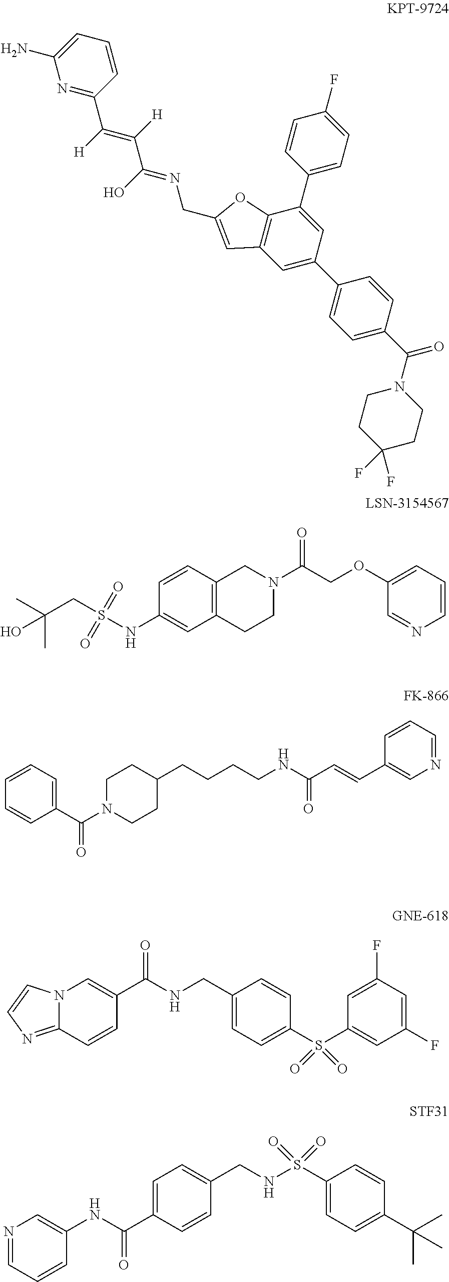 [SMILES: Nc1cccc(/C=C/C(O)=N/Cc2cc3cc(-c4ccc(C(=O)N5CCC(F)(F)CC5)cc4)cc(-c4ccc(F)cc4)c3o2)n1 ; CC(C)(O)CS(=O)(=O)Nc1ccc2c(c1)CCN(C(=O)COc1cccnc1)C2 ; O=C(/C=C/c1cccnc1)NCCCCC1CCN(C(=O)c2ccccc2)CC1 ; O=C(NCc1ccc(S(=O)(=O)c2cc(F)cc(F)c2)cc1)c1ccc2nccn2c1 ; CC(C)(C)c1ccc(S(=O)(=O)NCc2ccc(C(=O)Nc3cccnc3)cc2)cc1]

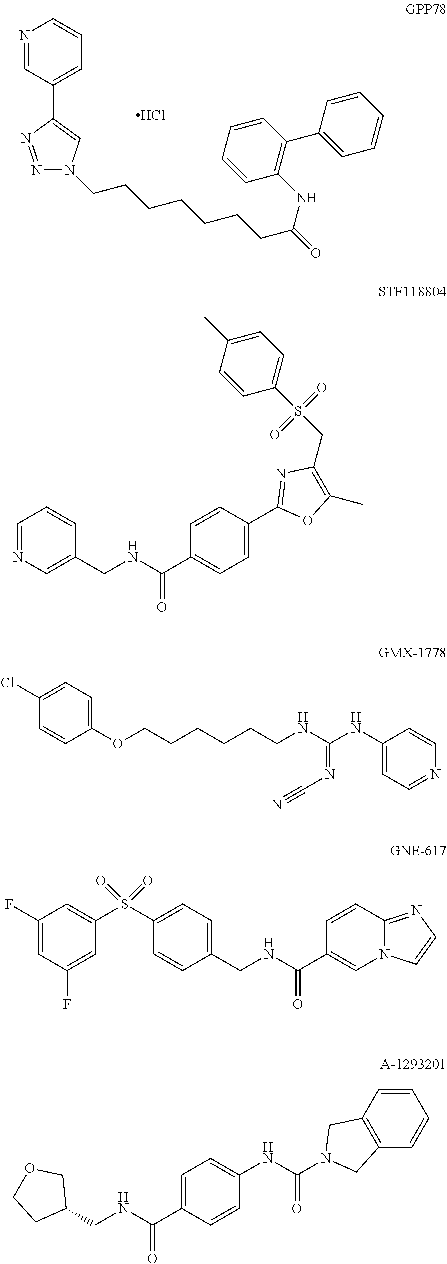 [SMILES: O=C(CCCCCCCn1cc(-c2cccnc2)nn1)Nc1ccccc1-c1ccccc1 ; Cl ; Cc1ccc(S(=O)(=O)Cc2nc(-c3ccc(C(=O)NCc4cccnc4)cc3)oc2C)cc1 ; N#C/N=C(\NCCCCCCOc1ccc(Cl)cc1)Nc1ccncc1 ; O=C(NCc1ccc(S(=O)(=O)c2cc(F)cc(F)c2)cc1)c1ccc2nccn2c1 ; O=C(NC[C@@H]1CCOC1)c1ccc(NC(=O)N2Cc3ccccc3C2)cc1]

Any cancer exhibiting a heightened level of PPM1D may be treated using various embodiments of the method of the invention. In various embodiments, the cancer is breast, ovarian, gastrointestinal, medulloblastoma or brain cancer. In various embodiments, the cancer may be a pediatric glioma.

As discussed further in Example 1, further NAD depleting treatments may increase the sensitivity of cancer cells with high levels of PPM1D to NAMPT inhibitors. Accordingly, in various embodiments, the method further comprises administering to the subject at least one additional nicotinamide adenine dinucleotide (NAD) depleting treatment. In various embodiments, the additional NAD depleting treatment is selected from the group consisting of administration of temozolomide, etoposide, irinotecan and radiation therapy.

Administration of supplemental nicotinamide may further increase the therapeutic index of NAMPT inhibitors with respect to cancers with elevated levels of PPM1D. Without wishing to be limited by theory, this may be because healthy cells are able to use the supplemental nicotinamide for the production of NAD while via the production of NAD through the NA salvage pathway while cancer cells cannot, as it has been found that elevated PPM1D blocks this pathway via NAPRT silencing. Accordingly, in various embodiments, the method, further comprises administering supplemental nicotinamide to the subject.

In various embodiments, the NAMPT inhibitor is administered in a pharmaceutical composition comprising at least one pharmaceutically acceptable excipient. In various embodiments the subject is a mammal. In various embodiments the subject is a human.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 1 μM and 10 μM in a mammal. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In certain embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., anti-fibrotic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of a disease or disorder contemplated in the invention. Using a wax/pH-sensitive polymer mix, a gastric insoluble composition may be obtained in which the active ingredient is entrapped, ensuring its delayed release.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the following Examples are here described.

### Cell Culture Materials and Techniques

hTert/E6/E7 immortalized human astrocytes were acquired from the lab of Dr. Timothy Chan, and have been previously characterized. Unless noted otherwise, astrocytes were grown in DMEM, high glucose (ThermoFisher Scientific/Gibco) plus 10% FBS (Gibco) as adherent monolayers. U2OS cells were purchased from ATCC, and were grown in DMEM, high glucose plus 10% FBS. MCF7 cells were grown in RPMI1640 (ThermoFisher Scientific/Gibco) with the addition of 10% FBS. HSJD-DIPG-007, HSJD-DIPG-008, and SU-DIPGs lines were all cultured in a Tumor Stem Media Base (DMEM/F12 and Neurobasal media) with the addition of growth factors: B27 supplement (Gibco/ThermoFisher), human EGF (Sigma), human FGF (Sigma), human PDGF (Sigma), heparin (Stemcell Technologies), and with or without the addition of nicotinic acid (Sigma), as indicated.

TABLE 1

| Name | Type | Sequence |
|---|---|---|
| PPM1D guide RNA top | gRNA oligo | SEQ ID NO: 1 |
| | | ACACCGTTGAGGGTATGACTACACCT |
| | | G |
| PPM1D guide RNA bottom | gRNA oligo | SEQ ID NO: 2 |
| | | AAAACAGGTGTAGTCATACCCTCAAC |
| | | G |
| PPM1D gDNA sequencing forward | primer | SEQ ID NO: 3 |
| | | GCATAGATTTGTTGAGTTCTGGG |
| PPM1D gDNA sequencing reverse | primer | SEQ ID NO: 4 |
| | | AGCCCTCTTATATCCTAAGTTTGG |
| PPM1D Site-directed mutagenesis | primer | SEQ ID NO: 5 |
| | | CCAGTCAAGTCACTCGAGGAGGATCC |
| | | ATGACCAAGGGTGAATTC |
| PPM1D Site-directed mutagenesis | primer | SEQ ID NO: 6 |
| | | GAATTCACCCTTGGTCATGGATCCTCC |
| | | TCGAGTGACTTGACTGG |
| NAPRT promoter bisulfite | primer | SEQ ID NO: 7 |
| sequencing forward | | CACCTCTGGTGACCAAGACC |
| NAPRT promoter bisulfite | primer | SEQ ID NO: 8 |
| sequencing reverse | | GTGGCCTGGTAGAGGTCAGT |
| NAPRT qPCR forward | primer | SEQ ID NO: 9 |
| | | CGAGAGGAGTTGGGTGACATCC |
| NAPRT qPCR reverse | primer | SEQ ID NO: 10 |
| | | CCTATGGCGCACTCCCTGTG |
| BAT26 forward | primer | SEQ ID NO: 11 |
| | | 6FAM-TGACTACTTTTGACTTCAGCC |
| BAT26 reverse | primer | SEQ ID NO: 12 |
| | | TCTGCATTTTAACTATGGCTC |
| D2S123 forward | primer | SEQ ID NO: 13 |
| | | 6FAM-AAACAGGATGCCTGCCTTTA |
| D2S123 reverse | primer | SEQ ID NO: 14 |
| | | GGACTTTCCACCTATGGGAC |

### CRISPR/Cas9 Genomic Editing and Plasmids

CRISPR/Cas9 genomic editing was performed in astrocytes using expression of both Cas9 (Addgene #43861) and a modified guide RNA (gRNA) construct (Addgene #43860). PPM1D gRNA sequences are available in Table 1 and were synthesized, annealed, and ligated into the gRNA plasmid. Both constructs were then co-transfected into astrocytes through nucleofection (Lonza), and the cells were incubated for 72 hours prior to harvest and isolation. Isolated clones were generated through a single cell dilution approach, and were grown up from individual wells of a 96-well plate. Clone screening for mutant PPM1D sequences and expression was performed using high resolution melt analysis screening methods and by western blotting as described below.

### Creation and Integration of Expression Constructs

An hWIP1 wild type plasmid (Addgene #28105). PPM1D was then subcloned from hWIP1 into a modified-phCMV1 expression construct creating PPM1D OE^{FL}. This construct was modified using site-directed mutagenesis, with the primers listed in Table 1, to introduce an R458fs mutation, creating PPM1D OE^{trnc.} All constructs were amplified in E. coli and purified using a MidiPrep kit (Qiagen), for nucleofection into cell lines as described above. Stable cell lines were selected with G418 (Gibco/ThermoFisher), and further isolated from single cell cultures. hWIP1 D314A phosphatase dead expression construct (Addgene #28106) was also amplified and purified as described above, and nucleofected into parental astrocytes prior to experimentation. A NAPRT expression construct was purchased from GenScript (OHu28558D) and amplified and purified as described above. Plasmid was nucleofected in PPM1D^{trnc.} astrocytes, selected with G418, and further isolated from single cell cultures.

### Western Blotting

Immunoblots were separated by SDS-PAGE and transferred to a PVDF membrane for analysis. All blots were blocked in 5% BSA (Gold Biotechnology) in 1×TBST (American Bio), and then were probed overnight at 4° c., with primary antibodies raised against: PPM1D (SCBT F-10 sc-376257, 1:1000), GAPDH (Proteintech group HRP-60004, 1:5000), Actin (ThermoFisher MA5-11869, 1:2000), γH2AX pS139 (CST 2577, 1:1000), NAPRT (Proteintech group 66159-1-Ig, 1:2000), NAMPT (CST 86634, 1:1000), pCHK2 T68 (CST 2197, 1:1000), H3K27M (CST 74829, 1:1000), or p53 (CST 9282, 1:1000). Blots were then washed with 1×TBST and incubated with HRP conjugated-anti-mouse (ThermoFisher 31432, 1:10,000) or anti-rabbit (ThermoFisher 31462, 1:10,000) secondary antibodies for 1 hour at room temperature (RT). Immunoblot exposure was carried out using Clarity Western ECL substrate (BioRad), and imaged on a ChemiDoc (BioRad) imaging system. Uncropped and unprocessed scans of all western blots shown are available in the Source Data file.

### In Vitro Chemical and IR Treatments

PPM1D^{trnc.} astrocytes were treated with 50 g/mL cycloheximide or 1 μM MG132 (both Sigma) for the indicated amount of time. Cells were then washed, pelleted, and lyzed for subsequent immunoblotting approaches, as described above. Quantification of immunoblot intensity was calculated using ImageJ software, and consisted of multiple (n=3) blots. Irradiation of cells was performed using an X-RAD KV irradiator (Precision X-ray), and treatment consisted of an unfractionated, 10 Gy dose. PPM1D inhibitor treatment with GSK2830371 (Selleckchem), consisted of 50 nM treatment, 24 hours prior to IR. FK866 (Selleckchem), GPP78 (Tocris Bioscience), STF118804 (Tocris Bioscience), STF31 (Tocris Bioscience), 5-azacytidine (Selleckchem), and Decitabine (Selleckchem) were dissolved in DMSO and used for treatment as indicated. Nicotinamide riboside (ChromaDex Inc.) and nicotinamide (Sigma) were dissolved in water while nicotinic acid (Sigma) was dissolved in PBS, prior to treatment alone or in combination with FK866, as indicated.

### γH2AX Foci Staining and Imaging

Astrocyte cell lines were seeded and incubated overnight, before radiation. Plates were then collected at indicated time points, fixed, permeabilized/blocked, and stained with primary and secondary antibodies for fluorescent imaging. Fixation was achieved with a 20 minute RT incubation in fixation buffer (4% paraformaldehyde and 0.02% TritonX100, in PBS). Cells were subsequently washed in 1×PBS, followed by a joint permeabilization and blocking step in incubation buffer (5% BSA and 0.5% TritonX100, in PBS) for 1 hour. Primary antibody raised against γH2AX pS139 (Millipore 05-636) was added at a dilution of 1:1000 in incubation buffer, and incubated overnight at 4° C. Plates were washed, followed by a 1 hour RT incubation with alexafluor-conjugated secondary antibodies (ThermoFisher A21425 or A11029, 1:10,000) and a nuclear dye, 1 g/mL Hoechst 33342 (Sigma), in secondary buffer (0.5% TritonX100, in PBS). Plates were again washed, and imaged in PBS using the Cytation3 imaging system (BioTek). Images were stitched using Gen5 v2.09 software (BioTek), and both foci and cell numbers were counted using CellProfiler image processing software.

### Drug Screen and Cellular Viability Measurements

In vitro cellular viability assessments of immortalized human astrocytes, MCF7, and U2OS cell lines were made using a previously described, high-content, microscopy platform developed by our group. In brief, cells were plated in a 96-well plate at a density of 2000 cells/well, and incubated overnight. Drug treatment or vehicle (0.5% DMSO) control was administered, and cells were incubated for 72-96 hours as indicated. Plates were then washed with PBS and fixed with ice-cold 70% ethanol for 2 hours at 4° C. After removal of ethanol, plates were again washed with PBS, and stained for 30 minutes at RT, with 1 g/mL Hoechst 33342 (Sigma). Cells were imaged using a Cytation3 imager (BioTek), and images were stitched and analyzed as described above. Viability assessments were made comparing drug treated to vehicle treated conditions. SU-DIPG and HSJD-DIPG-007 spheroid viability was assessed using CytoTox-Glo (Promega), according to the manufacturer's protocols. Spheroids were treated with FK866 for 120 hours before analysis using this method. IC₅₀ calculations were made using GraphPad Prism, by fitting data to an [inhibitor] vs response−variable slope four parameter nonlinear regression (as depicted in the representative figures).
siRNA Transfection and Viability Analysis
Individual NAPRT targeting siRNAs were ordered from Dharmacon Inc. (Horizon Discovery), with target sequences listed in Table 1. The panel of siRNAs used for synthetic lethal viability screening was hand-selected and ordered from Dharmacon Inc. and were provided in ON-TARGET plus mixtures, each containing up to four unique siRNAs per gene. 2×10⁵ astrocytes were reverse-transfected with different siRNAs (200 nM final concentration), using Lipofectamine RNAiMAX (Invitrogen), according to manufacturer's protocols. For individual siRNAs, cells were incubated for 72 hours, pelleted, and lyzed for immunoblotting. For the siRNA screen, cells were incubated for 24 hours and split to different condition plates, where they were incubated for an additional 24 hours. Cells were then treated with the described doses of FK866, and viability was assessed after 72 hours of drug treatment, using the image-based pipeline described above. Viability measurements were made for each siRNA, and normalized to FK866-untreated conditions.

### NAD Metabolome Quantification

The NAD metabolome was quantitatively analyzed using LC-MS/MS, using two separations on Hypercarb and 13C metabolite standards. Subsequent NAD level analyses were performed using a NAD/NADH Quantification kit (Sigma), as per the manufacturer's specifications.
Me/hME-DIP, Bisulfite Conversion, and Global 5-hmC Detection
Genomic DNA was purified using the Wizard Genomic DNA purification kit (Promega), and subsequently immunoprecipitated or bisulfite-converted. Immunoprecipitation assays were performed using Me-DIP and hMe-DIP kits (Active Motif), according to suggested protocols. Immunoprecipitated DNA was extracted with phenol/chloroform and analyzed using quantitative PCR (qPCR), as described below. Bisulfite conversion was performed via EpiMark Bisulfite Conversion kit (NEB). Modified DNA was then amplified using EpiMark Hot Start Taq DNA polymerase (NEB), with primers listed in Table 1, and purified with a PCR purification kit (Qiagen). Methylation was then assessed through Sanger-sequencing of the NAPRT promoter. Global 5-hydroxymethylcytosine levels were assessed via the Global 5-hmC quantification kit (Active Motif), according to manufacturer's protocols.
Quantitative PCR (qPCR)
mRNA transcripts were purified from cells using a RNAeasy kit (Qiagen) and subsequently reverse transcribed using a High Capacity cDNA reverse transcription kit (Applied Biosystems). PPM1D and NAPRT gene expression levels were assessed through qPCR with TaqMan fluorescent probes (all from Applied Biosystems): PPM1D (4331182), NAPRT (4351372), and Actin (4333762F), according to manufacturer's protocol. Expression level fold change was calculated via ΔΔCt comparison, using Actin as a reference gene. The NAPRT promoter region was quantitated via qPCR using Fast Start Universal SYBR Green Master with ROX (Roche), and primers listed in Table 1. All qPCR reactions were run on a StepOnePlus Real Time PCR system (Applied Biosystems).

### Infinium Methylation EPIC Array and Analysis

50-500 ng of genomic DNA was bisulfite-converted and analyzed for genome-wide methylation patterns using the Illumina Human EPIC Bead Array (850k) platform according the manufacturer's instructions. Data was processed and analyzed using Genome Studio v1.9 for NAPRT specific probes and methylation β-values were generated for all probes for downstream analyses. Global hypermethylation assessments were made using Limma R package of t-test model, with false discovery correction (FDR) and an absolute β-values threshold, to identify probes that reached significance in methylation differential between PPM1D mutant and wild samples (also known as significantly variable probes, or SVPs). Top 2% most variable probes lists were selected for based on variance and analyzed from the dataset, as described above, filtered for CpG island probes and delta β 0.2, and used for comparison to publicly available data which was processed similarly.

### Chromatin Immunoprecipitation (ChIP)

ChIP assays were performed using ChIP-IT Express kit (Active Motif), with a Rabbit IgG antibody (CST 2729) as an enrichment control. qPCR analysis for the NAPRT promoter was performed as described above. ChIP antibodies used: H3K4me1 (Abcam ab8895), H3K4me3 (CST 9751), H3K27me3 (CST 9733), and H3K27ac (Abcam, ab4729) at the manufacturer's recommended dilutions for ChIP.

### Animal Handling and In Vivo Studies

Astrocyte xenograft studies were performed in NOD scid gamma (NSG, NOD.Cg-Prkdc^{scid}Il2rg^{tm1Wj1}/SzJ female mice 3-4 weeks old) mice. For cell line xenografts, 5×10⁶ WT or PPM1D^{trnc.} astrocytes, stably expressing firefly luciferase (lentivirus-plasmids from Cellomics Technology; PLV-10003), were combined with Matrigel (Corning, 47743-722) in a total volume of 0.2 mL. Cell-Matrigel suspension was injected subcutaneously into both the right and left flanks of shaved NSG mice. Mice were randomly sorted into treatment groups, and tumor burden and growth were measured on a weekly basis, via BLI intensity, as described below. FK866 was solubilized in DMSO at a concentration of 80 mg/ml. Mice were then administered the drug intraperitoneally twice a day for 4 days, repeated weekly for 3 weeks at 20 mg/kg in 10% cyclodextrin. Treatment began after one month of growth. Tumors were harvested after completion of treatment, and mass for each tumor was measured. Serially transplanted xenografts were created via continuous transplantation of PPM1D^{trnc.} cell line xenografts in NSG mice. Subcutaneous flank injection with 5×10⁶ cells was performed with Matrigel as described above. Mice were sorted randomly into treatment groups, and tumor volume was measured using standard caliper-based techniques. Tumor volume was calculated as length×width²×0.52. U2OS xenograft studies were performed in athymic nude mice. 5×10⁶ cells were injected subcutaneously into the right flank of each animal and allowed to grow for 18 days before treatment. Mice were sorted randomly into treatment groups, and tumor burden was assessed through caliper measurement and volume calculations. FK866 was prepared and dosed as described above.

### Bioluminescent Imaging of Tumor Burden

Bioluminescence imaging (BLI) was performed using the IVIS Spectrum In Vivo Imaging System (PerkinElmer) according to the manufacturer's protocol. Images were taken on a weekly basis, and acquired 15 minutes post intraperitoneal injection with d-luciferin (150 mg/kg of animal mass). Quantification of BLI flux (photons/sec) was made through the identification of a region of interest (ROI) for each tumor, which was then circumscribed, background-corrected, and measured for BLI signal. Both right and left flank tumors were averaged together for each mouse, and then subsequently used for treatment group comparisons and analysis. All representative bioluminescent images were generated using a standard luminescent scale, and cropped to eliminate background objects.

### DIPG Expression Data

Data from the Pacific Pediatric Neuro-Oncology Consortium (PNOC) NCT02274987 study contained PPM1D and NAPRT expression levels from 29 newly diagnosed DIPG cases. RNA-sequencing was performed using Illumina HiSeq per the manufacturer's protocol, and was used to calculate transcript abundance. Pearson's Correlation r was calculated using GraphPad Prism. Data from HSJD-DIPG lines and additional DIPG model cell lines was acquired from a previously published dataset which was collated from Affymetrix Agilent and Illumina expression arrays and from RNASeq.

### Statistical Analysis and Significance

Unless otherwise described, data was analyzed on Microsoft Excel and GraphPad Prism software. Student's two-tailed T test for significance was used for comparisons between two groups and described as significant at *=p<0.05, **=p<0.01, * **=p<0.001, ****=p<0.0001. Mann-Whitney test was used to assess tumor growth curves, using the same significance denotations as above. Log rank (Mantel-Cox) test was used to assess significance in tumor delay as measured by Kaplan-Meier plot. All error bars shown are standard deviation of the mean, unless indicated otherwise.

### Example 1

### PPM1D Mutant Astrocytes are Sensitive to NAMPT Inhibitors

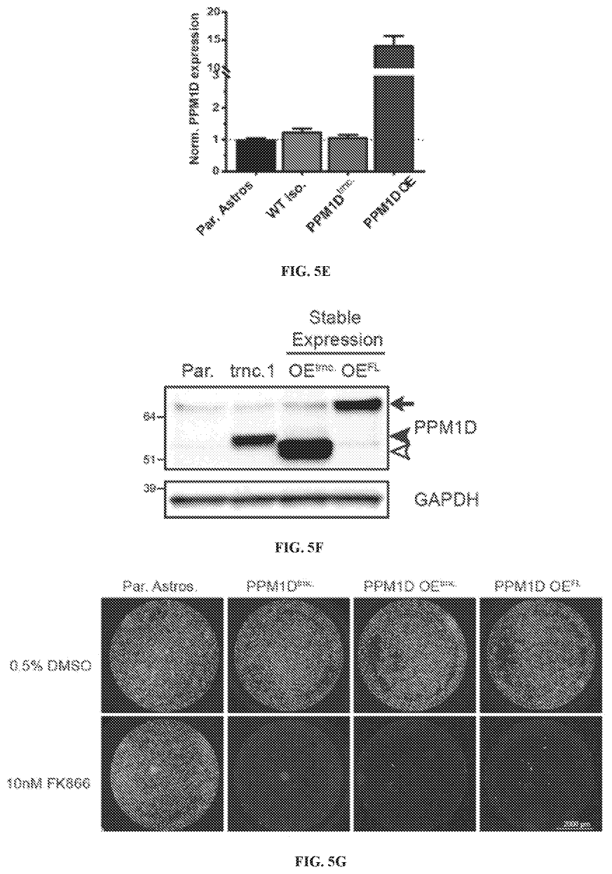

To develop PPM1D mutant models for subsequent biological investigations, we used CRISPR/Cas9 genomic editing to create isogenic immortalized human astrocytes harboring endogenous PPM1D truncation mutations (PPM1D^{trncs.}). The heterozygous, truncating mutations were introduced into exon 6 of the PPM1D locus, at C-terminal locations similar to those found in DIPGs (FIG. 1A). We then isolated single cell PPM1D^{trnc.} clones and confirmed the presence of frameshifting mutations that encode truncated PPM1D proteins (FIG. 5A). As expected, truncated PPM1D was highly expressed in mutant cells (FIG. 1B) and maintained a substantially longer half-life compared to the wild type (WT), full-length form of the protein (FIGS. 1C and 1D). The increased PPM1D protein stability correlated with enhanced phosphatase activity as seen by the active dephosphorylation of key PPM1D targets, γH2AX and pCHK2 (T68), measured by western blot (FIG. 5B) and γH2AX foci formation assays (FIG. 1E and FIG. 5C), after exposure to ionizing radiation (IR). Importantly, these differences were abolished by treatment with GSK2830371, a known inhibitor of PPM1D (FIG. 1F).

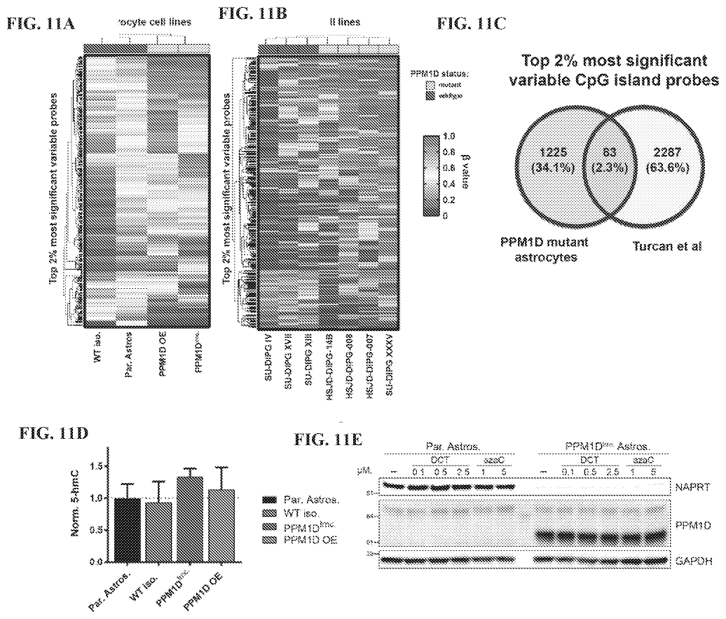

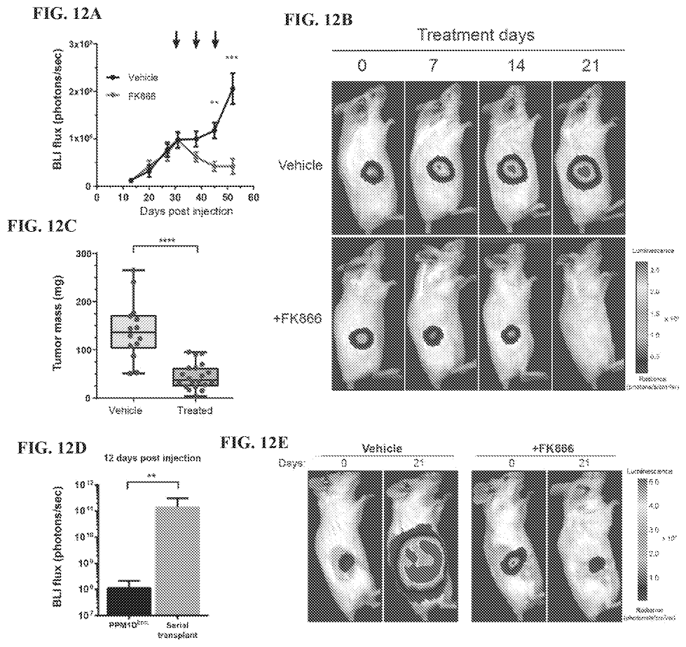

Given the role of PPM1D in DDR pathways, we performed a small molecule synthetic lethal screen with a panel of inhibitors against key DNA repair and metabolic proteins, using methodology described previously by our group. This screen identified a synthetic lethal interaction between PPM1D mutations and the nicotinamide phosphoribosyltransferase (NAMPT) inhibitor, FK866 (FIG. 1G; Table 2). This unexpected NAMPT inhibitor sensitivity was confirmed in three different PPM1D^{trnc.} cell lines (FIG. 1H), as well as by three structurally distinct NAMPT inhibitors: STF31, GPP78, and STF118804 (FIG. 11; FIG. 5D), corroborating our initial finding and establishing that this effect is a result of on-target inhibition of NAMPT activity. Furthermore, stable overexpression of either WT or mutant PPM1D in the parental astrocyte cell line (PPM1D OE^{FL} or OE^{trnc.}, respectively), was sufficient to confer FK866 synthetic lethality, confirming that this interaction is driven specifically by an increased total activity of PPM1D, and not a neomorphic role of the mutant protein (FIG. 1J; FIGS. 5E-5G). Additionally, expression of a phosphatase dead mutant (PPM1D D314A), did not result in FK866 sensitivity in our astrocyte models, further verifying the dependence on increased PPM1D activity for the induction of this synthetic lethality.

TABLE 2
Synthetic lethal drug screen compounds and IC₅₀ ratios.

| Drug Name | Company (catalog) | Target | ${IC}_{50} ( \frac{Par . Astros .}{PPM 1 {D}^{trncs}} )$ |
|---|---|---|---|
| FK866 | Selleckchem (S2799) | NAMPT | 9746.59 |
| Aphidicolin | Tocris (5736) | Topoisomerase 2 | 1.75 |
| TH287 | Selleckchem (S7631) | MTH1 | 1.52 |
| ETP 45658 | Tocris (4702) | DNApk | 1.51 |
| TMZ | Selleckchem (S1237) | DNA damage | 1.37 |
| SP2509 | Selleckchem (S7680) | LSD1 | 1.36 |
| Olaparib | Selleckchem (S1060) | PARP | 1.32 |
| MMS | Sigma (129925) | DNA damage | 1.28 |
| RITA | Selleckchem (S2781) | p53 | 1.27 |
| NU-7441 | Selleckchem (S2638) | DNApk, others | 1.26 |
| KU-55933 | Selleckchem (S1092) | ATM | 1.18 |
| Dexrazoxane | Selleckchem (S5651) | Blocks mitosis | 1.17 |
| TC52312 | Tocris (3038) | CHK1 | 1.13 |
| Lomustine | Selleckchem (S1840) | DNA damage | 1.10 |
| MMC | Selleckchem (S8146) | DNA damage | 1.08 |
| Bendamustine | Selleckchem (S1212) | DNA damage | 1.07 |
| MLM324 | Selleckchem (S7296) | JMJD2 | 1.02 |
| BEZ-235 | Selleckchem (S1009) | PI3K and mTOR | 1.01 |
| ATRN-19 | Atrin Pharm. | ATR | 1.01 |
| Irinotecan | Selleckchem (S2217) | Topoisomerase 1 | 1.01 |
| AZD6482 | Selleckchem (S1462) | DNApk, others | 1.00 |
| Etoposide | Selleckchem (S1225) | Topoisomerase 2 | 1.00 |
| G5K2879552 | Selleckchem (S7796) | LSD1 | 1.00 |
| BMN673 | Selleckchem (S7048) | PARP | 0.99 |
| Topotecan | Selleckchem (S1231) | Topoisomerase 1 | 0.99 |
| LSD1-C76 | Xcessbio (M66045-2s) | LSD1 | 0.98 |
| PIK 75 | Selleckchem (S1205) | DNApk, others | 0.97 |
| NCS | Sigma (N9162) | DNA damage | 0.94 |
| VE822 | Selleckchem (S7102) | ATR | 0.93 |
| MLN4924 | Selleckchem (S7109) | NAE (NHEJ) | 0.92 |
| Cyclophosphamide | Selleckchem (S2057) | DNA damage | 0.90 |
| PD 407824 | Tocris (2694) | CHK1/Wee1 | 0.83 |
| TC-S 7010 | Selleckchem (S1451) | Aurora A | 0.78 |
| AZD7762 | Selleckchem (S1532) | CHK1/2 | 0.77 |
| KU 0060648 | Selleckchem (S8045) | DNApk, others | 0.69 |
| MK-1775 | Selleckchem (S1525) | Wee1 | 0.63 |

Reduced NAD Levels in PPM1D^{trncs.} Drives NAMPT i Sensitivity

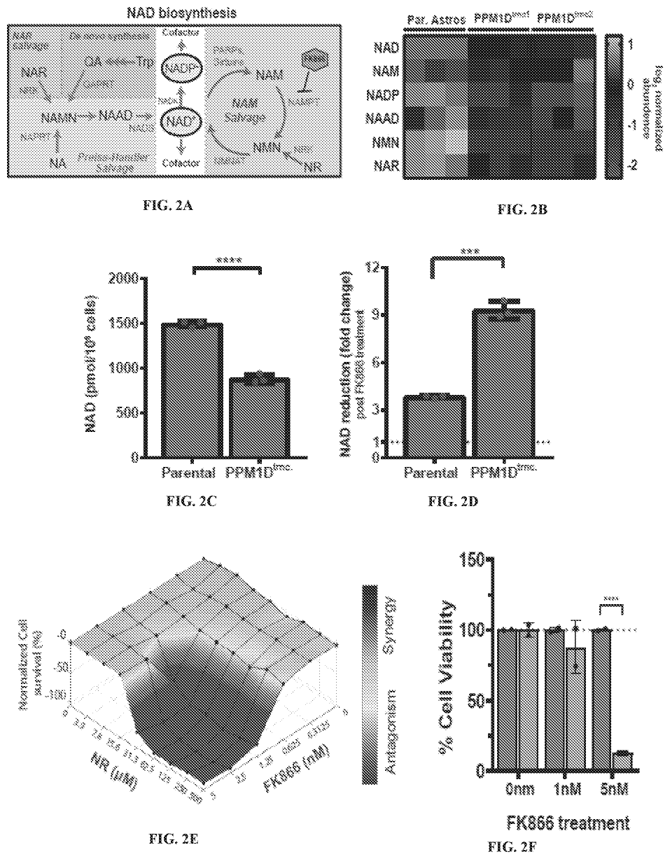

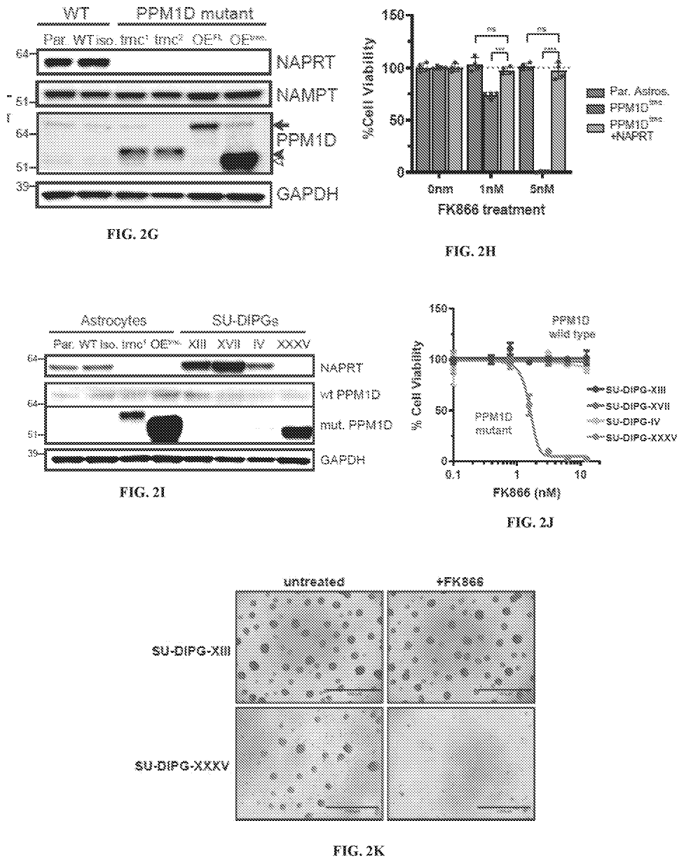

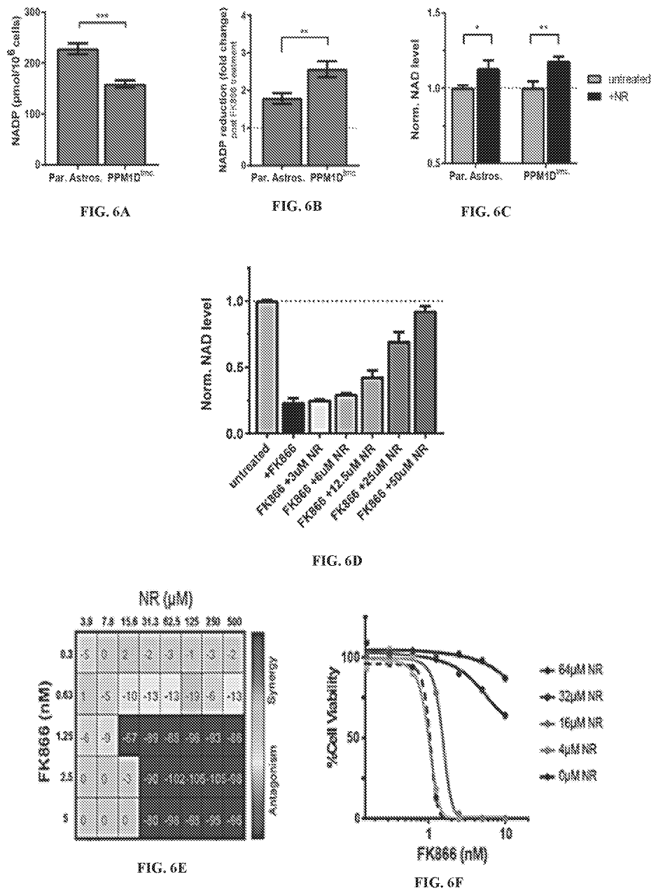

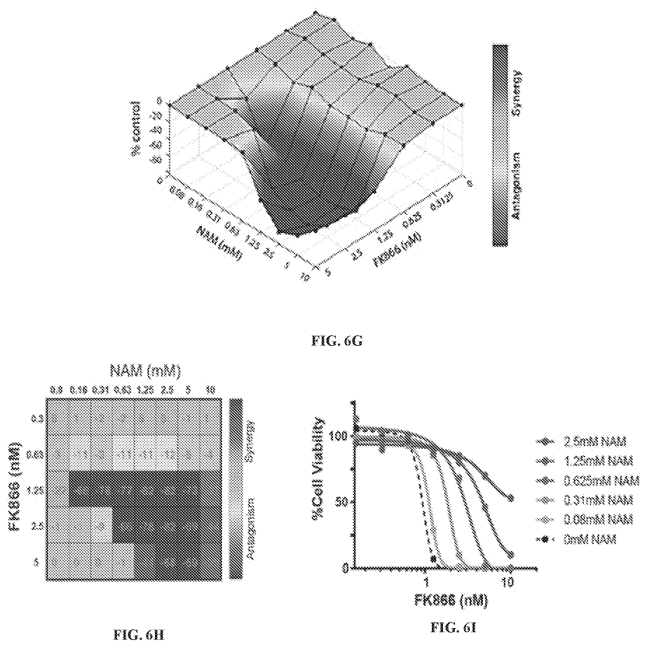

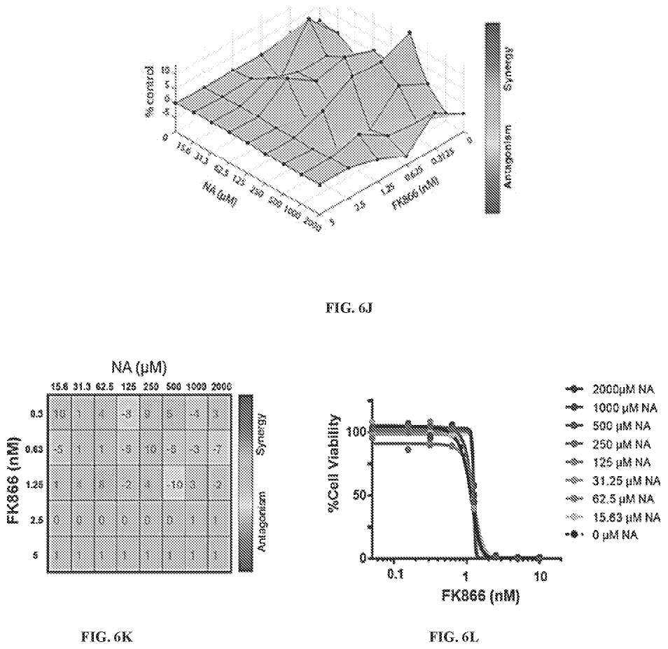

Next, we sought to investigate the molecular basis for mutant PPM1D-induced NAMPT inhibitor (NAMPTi) synthetic lethality. NAMPT catalyzes the rate-limiting step in the salvage of nicotinamide (NAM) to form nicotinamide adenine dinucleotide (NAD) (FIG. 2A). Thus, we wished to quantify the NAD metabolome, within our WT and PPM1D^{trnc.} astrocyte models to better understand potential variations in this important metabolic pathway. We found that PPM1D mutations induce a substantial depression of many NAD-related metabolites, including a significant reduction in NAD and NADP levels (FIGS. 2B, 2C; FIG. 6A). As maintenance of these two cofactors is important for cellular bioenergetics and proliferation, we examined the effect of NAMPT inhibition on the quantities of both NAD and NADP, as well as on cell viability. While cellular pools of both NAD and NADP dropped markedly in FK866-treated WT astrocytes, the decline was significantly greater in the PPM1D^{t}me. cells (FIG. 2D; FIG. 6B), indicating an enhanced dependence on NAMPT activity in the setting of mutant PPM1D. We then tested whether nicotinamide riboside (NR) could bypass NAMPT inhibition and thus, rescue the levels of NAD in PPM1D^{trnc.} astrocytes. Indeed, NR treatment sufficiently increased basal NAD levels (FIG. 6C, and FIG. 6D), and completely mitigated the cytotoxic effects of FK866 in PPM1D^{trnc.} cells (FIG. 2E; Supplementary FIG. 6E, and FIG. 6F). Similar results were found upon exogenous treatment of NAM, which strongly antagonized FK866 cytotoxicity in PPM1D^{trnc.} cells (FIGS. 6G-6I). Interestingly, exogenous treatment with NA did not prevent FK866-induced cell death, indicating a potential metabolic defect in the Preiss Handler salvage pathway (FIG. 6J-6L). Taken together, these data suggest that mutant PPM1D induces a depression of the NAD metabolome and especially NAD levels, which can be further potentiated by NAMPT inhibition, resulting in the selective killing of PPM1D mutant cells.

### PPM1D Mutant DIPG Models Silence NAPRT Gene Expression

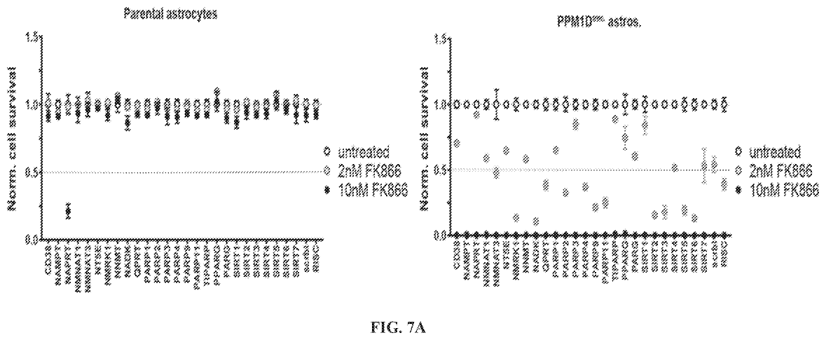

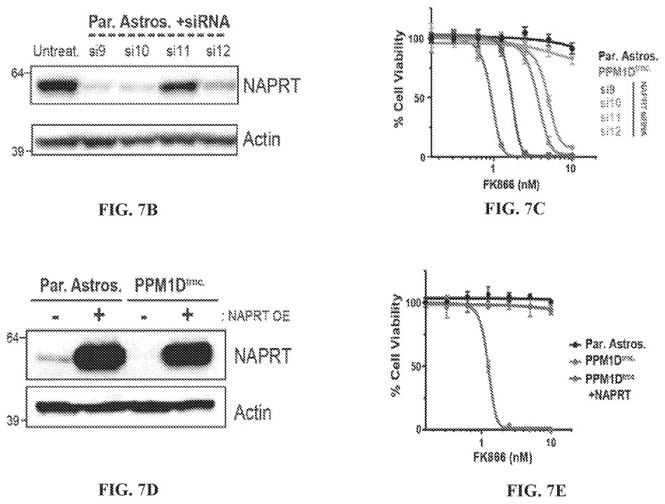

To understand the underlying cause of NAD depletion in PPM1D^{trnc.} cells, we performed a focused synthetic lethal siRNA screen in our isogenic astrocytes, targeting key enzymes involved in NAD synthesis and consumption pathways. Using FK866 sensitivity as an endpoint, the goal was to identify genes whose loss phenocopies the synthetic lethal interaction previously identified between mutant PPM1D and NAMPT inhibition. From this screen, we found that siRNA-mediated knockdown of nicotinic acid phosphoribosyltransferase (NAPRT) induced profound sensitivity of the parental astrocyte cell line to FK866 treatment (FIG. 2F; FIG. 7A). Additional NAPRT siRNAs were used to confirm these findings and further revealed a strong correlation between the degree of NAPRT knockdown and FK866 sensitivity (FIG. 7B, FIG. 7C). NAPRT plays a complementary role to NAMPT in the production of NAD, and previous studies have inversely correlated NAPRT expression with NAMPT inhibitor sensitivity. Surprisingly, we found that NAPRT protein expression was undetectable in our PPM1D^{trnc.} and PPM1D overexpressing (OE^{FL} and OE^{trnc.}) cell lines (FIG. 2G). To determine if this critical deficiency resulted in NAMPT inhibitor sensitivity, we reintroduced NAPRT into PPM1D^{t}me. cells. Stable, ectopic expression of NAPRT completely rescued the cytotoxicity caused by NAMPT inhibition, and mirrored the resistance found commonly in WT cells (FIG. 2H; FIG. 7D, 7E). Collectively, these findings suggest that mutant PPM1D drives a loss of NAPRT expression, which ultimately induces profound NAMPT inhibitor sensitivity.

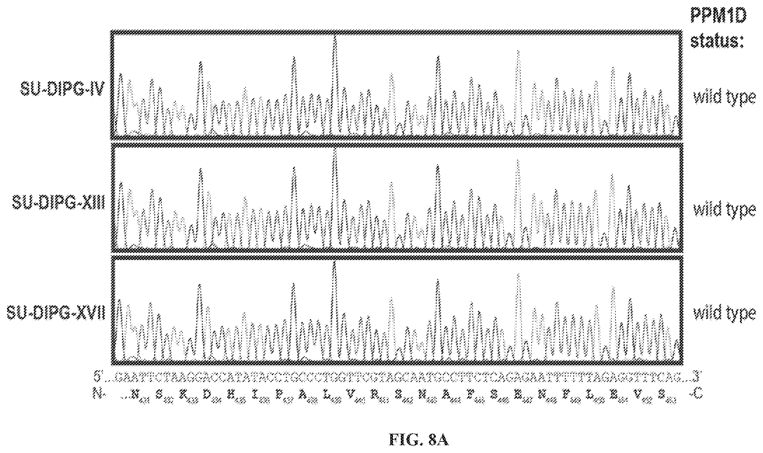

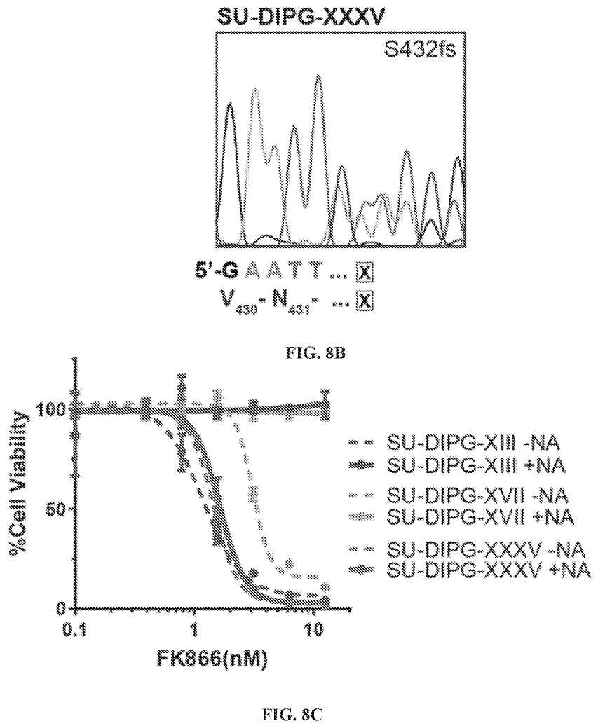

To complement our work in immortalized, normal human astrocytes, we then tested whether our findings could be recapitulated in more clinically relevant tumor models. To this end, we examined NAPRT expression in a collection of previously described, patient-derived DIPG spheroid cultures. One of these DIPG lines, SU-DIPG-XXXV, contained a S432fs mutation in PPM1D (FIG. 8A, FIG. 8B), and prominently expressed a hyperstable, truncated form of the protein (FIG. 2I). Similar to the PPM1D^{t}me. astrocytes, we found that SU-DIPG-XXXV also completely lacked NAPRT gene expression. This deficiency was unique in the DIPG cell panel as the remaining WT lines maintained high levels of NAPRT expression. Consistent with our findings in immortalized astrocytes, SU-DIPG-XXXV was also extremely sensitive to FK866 treatment (FIGS. 2J and 2K) with cytotoxic doses in the low, single-digit nanomolar range. In contrast, the three WT DIPG lines were resistant to FK866 treatment, highlighting the dependence of NAMPT inhibitor sensitivity on PPM1D mutation status. Notably, culturing these DIPG cell lines in growth media devoid of nicotinic acid (NA) induced a strong sensitivity to FK866 in all SU-DIPG spheroid cultures tested (FIG. 8C), confirming the importance of alternative NAD biosynthesis pathways such as NA salvage, in mediating NAMPT inhibitor synthetic lethality in gliomas.

### Epigenetic Events Silence NAPRT Expression in PPM1D Mutant Models

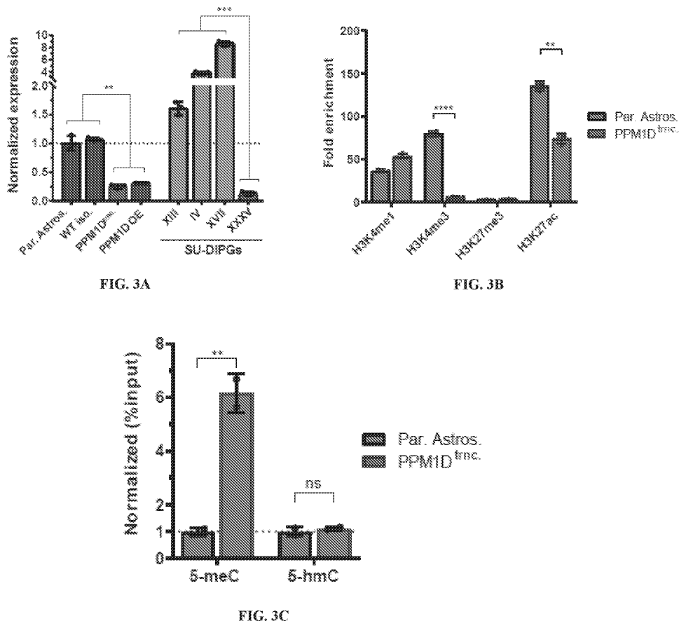

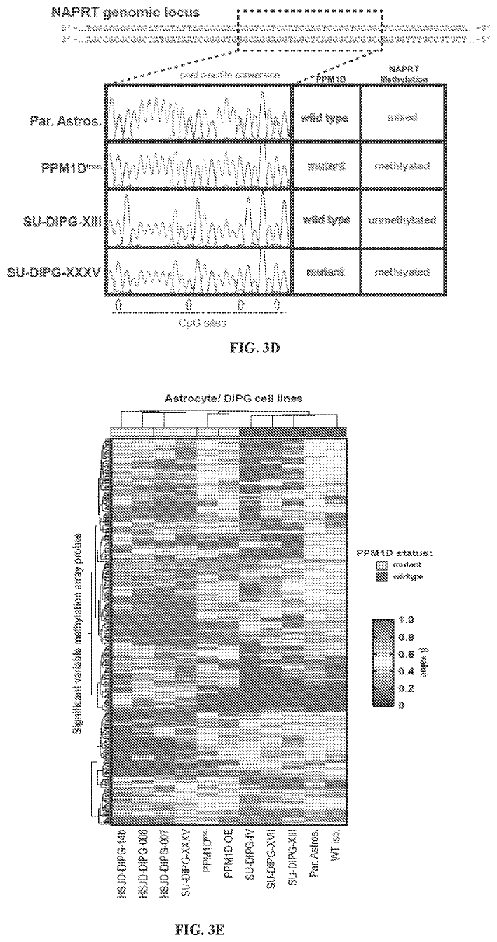

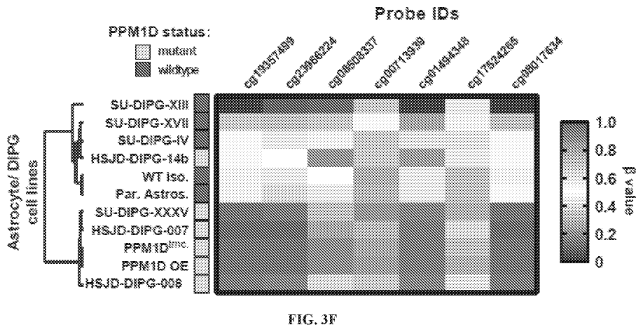

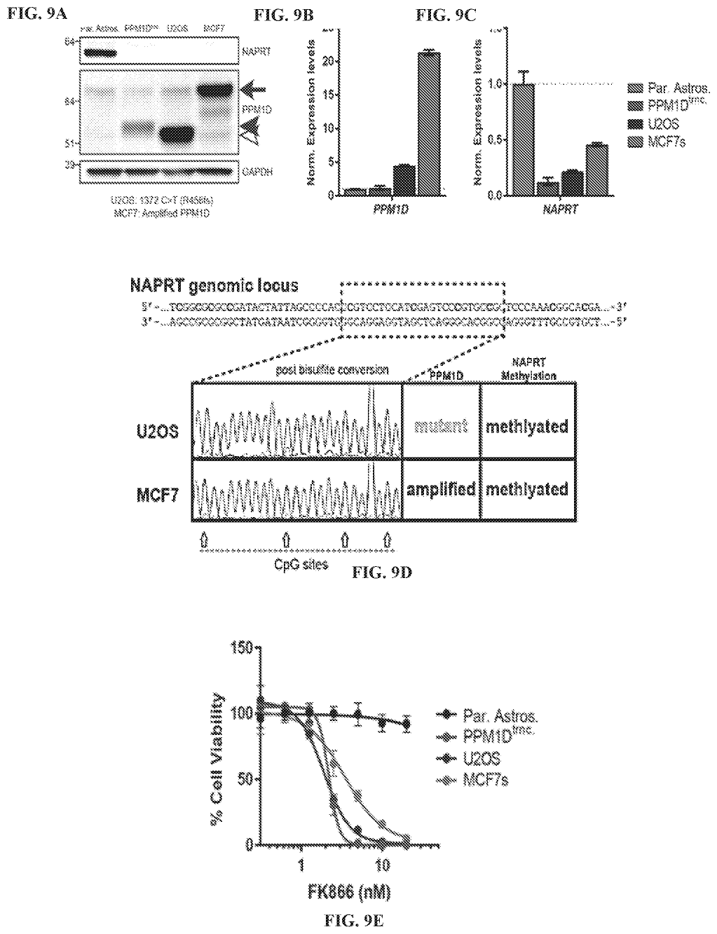

Next we sought to identify the mechanism by which mutant PPM1D suppresses NAPRT expression. While NAPRT mRNA was highly expressed in WT DIPG lines (SU-DIPG-IV, XIII, and XVII), NAPRT transcript levels were found to be significantly depressed in all PPM1D mutant astrocyte and DIPG models tested (PPM1D^{trnc.}, PPM1D^{OE}, and SU-DIPG-XXXV) (FIG. 3A), indicating the presence of a conserved transcriptional repression of the NAPRT gene. As transcriptional silencing is often controlled by epigenetic factors, we next examined the occupancy of different histone marks at the NAPRT promoter in WT and PPM1D mutant astrocytes. Using chromatin immunoprecipitation (ChIP), we found that transcriptional repression of NAPRT in PPM1D mutant cells correlated with a substantial loss in key activating chromatin marks, H3K4me3 and H3K27ac (FIG. 3B). It has previously been shown that a loss of occupancy of H3K4me3 and H3K27ac can result in an increase in site-specific DNA methylation. Additionally, the NAPRT promoter resides within a CpG island that is prone to de novo DNA methylation. Thus, we considered the possibility that mutant PPM1D induces silencing of the NAPRT gene by regulating DNA methylation at its promoter. To test this hypothesis, we immunoprecipitated and quantified methylated and hydroxymethylated cytosine bases from within the NAPRT promoter, using Me-DIP and hMe-DIP assays respectively. From this work we detected a prominent increase in DNA methylation, but not hydroxymethylation, at the NAPRT promoter in PPM1D^{trnc.} astrocytes (FIG. 3C). This finding was further confirmed with bisulfite conversion and sequencing of our astrocyte and DIPG models, which revealed extensive NAPRT promoter hypermethylation in all PPM1D mutant cell lines (FIG. 3D). To ascertain if this effect was specifically limited to DIPG and astrocyte models, we validated our results in the osteosarcoma cell line, U2OS (R458fs), as well as the breast cancer cell line MCF7 (PPM1D amplification), both which contain endogenous PPM1D alterations (FIGS. 9A and 9B). Similar to the PPM1D^{trnc.} astrocytes, we found substantial gene silencing of NAPRT transcription in U2OS and MCF7 cells, which corresponded with extensive hypermethylation of the NAPRT promoter (FIGS. 9C and 9D). Further, both cell lines displayed a strong sensitivity to FK866 treatment, which was comparable to PPM1D^{trnc.} astrocytes and the other described PPM1D mutant DIPG models (FIG. 9E).

### PPM1D Mutations Promote Global CpG Island Hypermethylation

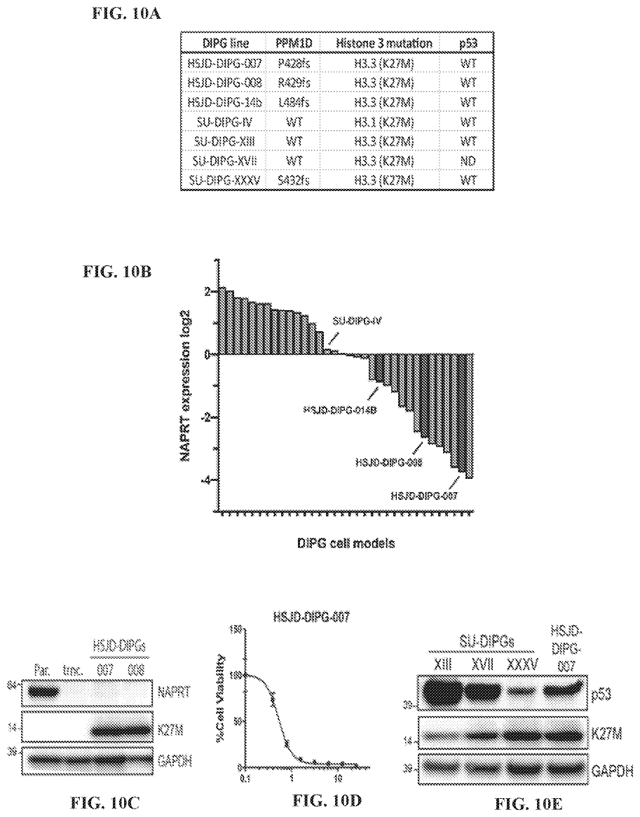

Next, we investigated whether mutant PPM1D-induced NAPRT gene silencing is a focal event or part of a more global phenomenon. Whole genome methylation profiling was performed on our entire panel of WT and PPM1D mutant cell lines, as well as on three additional PPM1D mutant DIPG lines: HSJD-DIPG-007, HSJD-DIPG-008, and HSJD-DIPG-14b; all of which maintain reduced expression of NAPRT (27) and/or sensitivity to FK866 treatment (FIGS. 10A-10D). Methylation results from the Illumina Human EPIC Bead Array (850k) revealed a substantial increase in CpG island hypermethylation across all PPM1D mutant cell lines tested. Of the 390 most significant variable probes (SVPs), 287 (74%) were hypermethylated in PPM1D mutant lines (PPM1D^{trnc.}, PPM1D^{OE}, SU-DIPG-XXXV, HSJD-DIPG-007, HSJD-DIPG-008, and HSJD-DIPG-14b), compared to only 103 (26%) hypermethylated in WT cell lines (FIG. 3E). In addition, individual probes within the NAPRT locus were subsequently identified and analyzed from this data set. All seven sites residing within the CpG island promoter region of NAPRT were heavily methylated in PPM1D mutant astrocytes and DIPG cultures, and bivariate correlational analysis clustered 5 of 6 mutant cells separately from tested WT lines (FIG. 3F). Interestingly, despite a lower overall degree of methylation within the NAPRT promoter in HSJD-DIPG-14b, this line did still exhibit hypermethylation across the SVPs described previously, and clustered similarly to the other PPM1D mutant lines upon whole genome methylation analysis. Of note, all DIPG lines tested harbored endogenous histone 3 K27M mutations (FIGS. 10A and 10E), which often co-occur with PPM1D truncating mutations in tumor samples. Despite previous reports linking H3.1 or H3.3 K27M mutations to global DNA hypomethylation, our results suggest that truncation alterations in PPM1D may in fact overcome this effect, and instead drive the hypermethylation of genomic CpG islands.

IDH1 R132H mutant gliomas famously exhibit a glioma-associated CpG island methylator phenotype (or G-CIMP), which arises from the competitive inhibition of DNA-demethylating TET proteins by the oncometabolite 2-HG. To understand if the hypermethylation events observed in our PPM1D mutant DIPG models paralleled those found in IDH1 mutant cell lines, we analyzed the top 2% of significantly variable CpG island methylation array probes, for comparison to a previously published IDH1 mutant data set (FIGS. 11A and 111B) While we identified a similar percentage of hypermethylated probes in the PPM1D- and IDH1 mutant cell lines compared to their parental astrocyte controls (79.4% and 63.9%, for PPM1D mutant- and IDH1 mutant astrocytes, respectively) we found surprisingly little over-lap between the two engineered mutant lines (FIG. 11C). Further, examination of global 5-hydroxymethylcytosine (5-hmC), a product of TET enzymatic activity, found no significant difference in 5-hmc levels between WT and PPM1D mutant astrocytes, indicating a distinct mechanism may be driving the development of genomic hypermethylation in these mutant cell lines (FIG. 11D). Lastly, treatment of PPM1D^{trnc.} cells with the DNA demethylating agents decitabine (DCT) and azacytidine (azaC) failed to reverse the gene silencing of NAPRT in these cells, further differing our results from previous studies in IDH1 mutant cell lines (FIG. 11E). Overall, these findings demonstrate that PPM1D mutations drive a unique pattern of global DNA methylation, distinct from that found in IDH1 mutant gliomas, which is associated with CpG island hypermethylation and NAPRT gene silencing.

### NAMPTi s are Efficacious In Vivo Against PPM1D^{mut} Xenografts

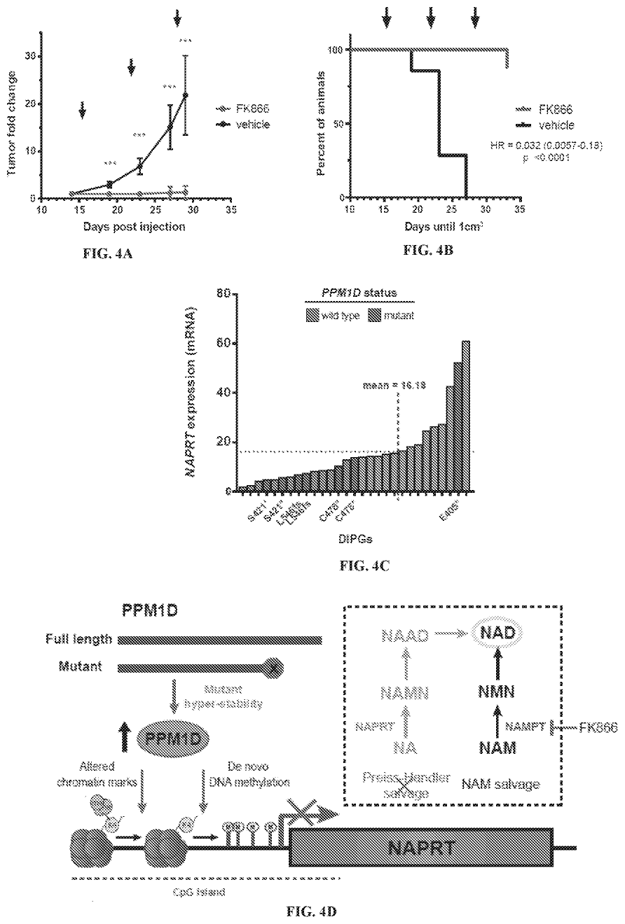

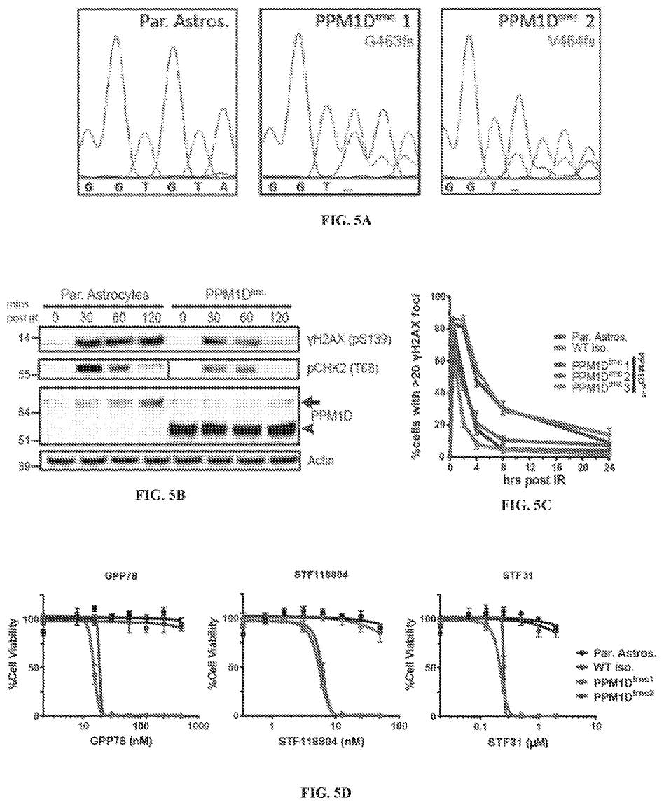

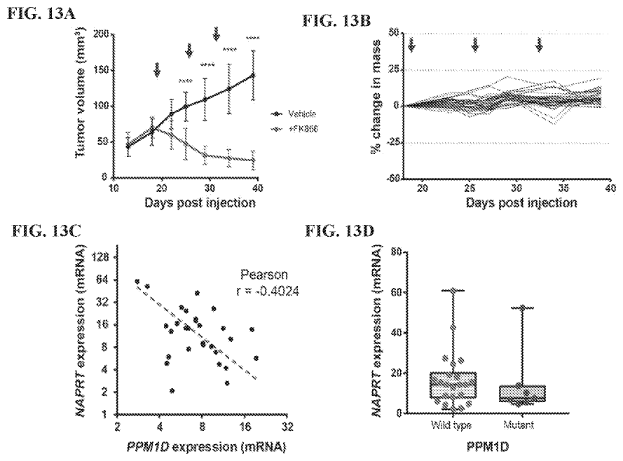

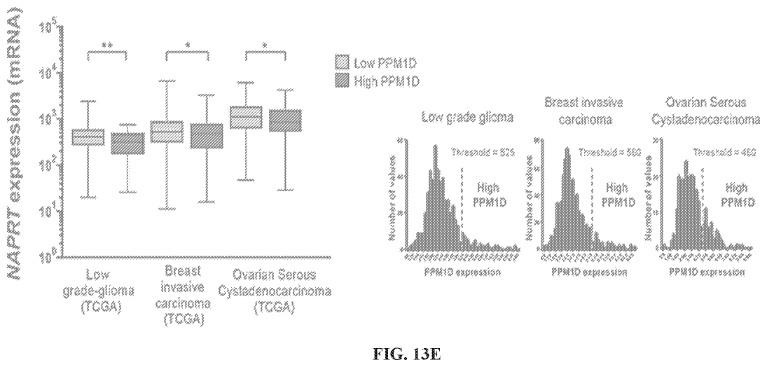

Next, we tested whether mutant PPM1D-induced NAMPT inhibitor sensitivity could be recapitulated in vivo. We subcutaneously injected both parental and PPM1D^{trnc.} cells into NOD scid gamma (NSG) mice and monitored tumor growth using bioluminescence imaging (BLI). While parental astrocytes failed to form tumors after 6 months, flank injection of PPM1D^{trnc.} astrocytes resulted in tumor formation within 30 days. Remarkably, treatment of these mice with FK866 induced a rapid reduction in tumor burden (fold change=4.93, p=0.0003 by Mann-Whitney U test) after three weeks (FIGS. 12A and 12B). These data correlated with substantially lower (fold change=3.1, p<0.0001 by Mann-Whitney U test) final tumor mass after treatment with FK866 versus vehicle alone (FIG. 12C). As the size and growth rate of PPM1D^{trnc.} xenografts limit the use of alternative measurement techniques, we created a serially-transplanted, PPM1D mutant astrocyte xenograft model. These PPM1D mutant xenografts form measurable tumors within 12 days of flank injection (FIG. 12D) and grow rapidly, allowing direct tumor volumes to be assessed. Treatment of these mice with FK866 greatly reduced the overall tumor size (fold change=17.1, p<0.0002 by Mann-Whitney U test), as measured by both calipers and BLI, (FIG. 4A; FIG. 12E), and significantly delayed tumor growth (p<0.0001 by Log rank (Mantel-Cox) test) compared to a vehicle control (FIG. 4B). Similar results were obtained in U2OS cell line xenografts, which again displayed significant sensitivity to FK866 treatment (fold change=5.86, p<0.0001 by Mann-Whitney U test) (FIG. 13A). Importantly, as NAMPT inhibitors have been associated with dose-related toxicities, the health and body mass of all mice on study were tracked throughout the dosing schedule, during which time we detected no significant differences in body mass between the treatment groups (FIG. 13B). Overall, our data strongly support the synthetic lethality seen with FK866 in vitro, and demonstrate the potential efficacy of NAMPT inhibitors for treatment of PPM1D mutant tumors.

Finally, using gene expression data from within a cohort of DIPG biopsy specimens (31), we identified a strong inverse correlation between PPM1D and NAPRT mRNA levels (FIG. 13C), as well as a trend of decreased NAPRT expression in known PPM1D mutant tumor samples (FIG. 4C; FIG. 13D). In parallel, we analyzed publicly available patient-derived cancer gene expression data from cBioPortal across tumor subtypes in which PPM1D is often found amplified, including brain, breast, and ovary. From this, we identified a trend of statistically significant differences in NAPRT expression between PPM1D low and high expressing tumors (FIG. 13E), providing additional validation across a diverse set of malignancies that associates expression of this oncogene with a potentially actionable and druggable target.

Altogether, our results establish a previously unknown role for PPM1D mutations as drivers of global DNA methylation, leading to NAPRT gene silencing. NAPRT catalyzes the first step in the Preiss-Handler NA salvage pathway to produce NAD. Thus, mutant PPM1D-induced silencing of NAPRT leads to a depression of the NAD metabolome. Loss of NAPRT necessitates a complete reliance of PPM1D mutant cells on other NAD-generating pathways for survival, principally the NAM-salvage pathway mediated by NAMPT. As a result, PPM1D mutant cells can be selectively targeted and killed with NAMPT inhibitors (FIG. 4D). Additionally, NAMPT inhibitor synthetic lethality was observed in an assorted panel of cells expressing high levels of both truncated or full-length PPM1D. This finding suggests broad clinical applicability, since PPM1D is amplified or over-expressed in a diverse range of cancers.

NAMPT inhibitors have been tested in clinical trials, although the lack of a prognostic biomarker, as well as dose-limiting hematologic toxicities, have stymied their further advancement into the clinic. Our study reveals a clinically-relevant biomarker, PPM1D mutations, which can be used for molecularly-informed personalized treatment of patients using NAMPT-inhibitor based therapeutic strategies. Furthermore, previous studies suggest that numerous DNA damaging agents, such as temozolomide and radiation therapy, also deplete cellular levels of NAD. As these agents are commonly used to treat tumors that harbor PPM1D mutations (e.g., DIPG), they could be combined with NAMPT inhibitors to further enhance tumor-selective cytotoxicity. Recent reports suggest that co-administration of NA can mitigate NAMPT inhibitor-associated hematologic toxicity via the production of NAD through the NA salvage pathway. Based on our observations that mutant PPM1D blocks this pathway via tumor-specific NAPRT silencing, NA supplementation may be an effective approach to further enhance the therapeutic index associated with NAMPT inhibition. Finally, our results reveal a unique pattern of CpG island hypermethylation events, specifically in DIPGs. This finding is reminiscent yet biologically distinct from that associated with IDH1 2 mutations in adult gliomas. Overall, our work demonstrates a completely independent route by which tumor-associated mutations can drive global DNA hypermethylation events, and sheds additional light on the molecular consequences of aberrant methylation in glioma biology.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating cancer in a subject, the method comprising
administering to the subject at least one nicotinamide phosphoribosyltransferase (NAMPT) inhibitor, thereby treating the cancer,
wherein protein phosphatase Mg²⁺/Mn²⁺ dependent 1D (PPM1D) is elevated in a biopsy sample obtained from the cancer in the subject.

2. The method of claim 1, further comprising detecting an elevated level of PPM1D relative to a reference level, in a cancer cell sample obtained from the subject.

3. The method of claim 1, wherein the cancer comprises one or more mutations in the PPM1D gene.

4. The method according to claim 1, wherein PPM1D comprises a C-terminal truncation mutation.

5. The method according to claim 1, wherein the at least one NAMPT inhibitor is selected from the group consisting of OT-82, KPT-9274, FK866, GNE-618, LSN-3154567, STF31, GPP78, and STF118804.

6. The method according to claim 1, wherein the cancer is breast, ovarian, gastrointestinal, brain cancer, medulloblastoma or pediatric glioma.

7. The method according to claim 1, further comprising administering to the subject at least one additional nicotinamide adenine dinucleotide (NAD) depleting treatment.

8. The method of claim 7, wherein the additional NAD depleting treatment is selected from the group consisting of temozolomide, etoposide, irinotecan and radiation therapy.

9. The method according to claim 1, further comprising administering supplemental nicotinamide to the subject.

10. The method according to claim 1, wherein an effective amount of the NAMPT inhibitor is administered to the subject in a pharmaceutical composition comprising at least one pharmaceutically acceptable excipient.

11. The method according to claim 1, wherein the subject is a mammal.

12. The method according to claim 1, wherein the subject is a human.

13. A method of treating cancer in a subject having elevated PPM1D in a biopsy obtained from said subject, the method comprising administering to said subject an effective amount of a NAMPT inhibitor.

14. A method of treating cancer in a subject having elevated PPM1D, the method comprising:
detecting in a cancer cell sample obtained from the subject an elevated level of PPM1D relative to a reference level; and
administering to said subject an effective amount of a NAMPT inhibitor.

15. The method according to claim 13, further comprising detecting in a cancer cell sample obtained from the subject an elevated level of PPM1D relative to a reference level.

16. The method according to claim 13, wherein the cancer comprises one or more mutations in the PPM1D gene.

17. The method according to claim 14, wherein the cancer comprises one or more mutations in the PPM1D gene.

18. The method according to claim 13, wherein the at least one NAMPT inhibitor is selected from the group consisting of OT-82, KPT-9274, FK866, GNE-618, LSN-3154567, STF31, GPP78, and STF118804.

19. The method according to claim 14, wherein the at least one NAMPT inhibitor is selected from the group consisting of OT-82, KPT-9274, FK866, GNE-618, LSN-3154567, STF31, GPP78, and STF118804.

20. The method according to claim 14, wherein the cancer is breast, ovarian, gastrointestinal, brain cancer, medulloblastoma or pediatric glioma.


SEQUENCE LISTING (16 sequences)

SEQ ID NO: 1 - DNA, 27 bp, Artificial Sequence
  gRNA oligo
acaccgttgagggtatgactacacctg

SEQ ID NO: 2 - DNA, 27 bp, Artificial Sequence
  gRNA oligo
aaaacaggtgtagtcataccctcaacg

SEQ ID NO: 3 - DNA, 23 bp, Artificial Sequence
  primer
gcatagatttgttgagttctggg

SEQ ID NO: 4 - DNA, 24 bp, Artificial Sequence
  primer
agccctcttatatcctaagtttgg

SEQ ID NO: 5 - DNA, 44 bp, Artificial Sequence
  primer
ccagtcaagtcactcgaggaggatccatgaccaagggtgaattc

SEQ ID NO: 6 - DNA, 44 bp, Artificial Sequence
  primer
gaattcacccttggtcatggatcctcctcgagtgacttgactgg

SEQ ID NO: 7 - DNA, 20 bp, Artificial Sequence
  primer
cacctctggtgaccaagacc

SEQ ID NO: 8 - DNA, 20 bp, Artificial Sequence
  primer
gtggcctggtagaggtcagt

SEQ ID NO: 9 - DNA, 22 bp, Artificial Sequence
  primer
cgagaggagttgggtgacatcc

SEQ ID NO: 10 - DNA, 20 bp, Artificial Sequence
  primer
cctatggcgcactccctgtg

SEQ ID NO: 11 - DNA, 21 bp, Artificial Sequence
  primer
tgactacttttgacttcagcc

SEQ ID NO: 12 - DNA, 21 bp, Artificial Sequence
  primer
tctgcattttaactatggctc

SEQ ID NO: 13 - DNA, 20 bp, Artificial Sequence
  primer
aaacaggatgcctgccttta

SEQ ID NO: 14 - DNA, 20 bp, Artificial Sequence
  primer
ggactttccacctatgggac

SEQ ID NO: 15 - protein, 491 aa, Homo sapiens [sequence omitted]

SEQ ID NO: 16 - protein, 605 aa, Homo sapiens
MAGLYSLGVSVFSDQGGRKYMEDVTQIVVEPEPTAEEKPSPRRSLSQPLPPRPSPAALPGGEVSGKGPAVAAREARDPLPDAGASPAPSRCCRRRSSVAFFAVCDGHGGREAAQFAREHLWGFIKKQKGFTSSEPAKVCAAIRKGFLACHLAMWKKLAEWPKTMTGLPSTSGTTASVVIIRGMKMYVAHVGDSGVVLGIQDDPKDDFVRAVEVTQDHKPELPKERERIEGLGGSVMNKSGVNRVVWKRPRLTHNGPVRRSTVIDQIPFLAVARALGDLWSYDFFSGEFVVSPEPDTSVHTLDPQKHKYIILGSDGLWNMIPPQDAISMCQDQEEKKYLMGEHGQSCAKMLVNRALGRWRQRMLRADNTSAIVICISPEVDNQGNFTNEDELYLNLTDSPSYNSQETCVMTPSPCSTPPVKSLEEDPWPRVNSKDHIPALVRSNAFSENFLEVSAEIARENVQGVVIPSKDPEPLEENCAKALTLRIHDSLNNSLPIGLVPTNSTNTVMDQKNLKMSTPGQMKAQEIERTPPTNFKRTLEESNSGPLMKKHRRNGLSRSSGAQPASLPTTSQRKNSVKLTMRRRLRGQKKIGNPLLHQHRKTVCVC